(19) United States
(12) Patent Application Publication
(10) Pub. No.: US 2026/0315585 A1
(43) Pub. Date: Oct. 15, 2026

(54) GLYCOPROTEIN ANTIGENS FOR HIV VACCINES

(71) Applicants: University of Washington, Seattle, WA (US); STICHTING AMSTERDAM UMC, Amsterdam (NL)

(72) Inventors: Neil P. KING, Seattle, WA (US); Yoann ALDON, Seattle, WA (US); Rogier SANDERS, HV Amsterdam (NL); Robert J. RAGOTTE, Seattle, WA (US); Basile I.M. WICKY, Seattle, WA (US); Lukas F. MILLES, Seattle, WA (US); Naveen JASTI, Seattle, WA (US)

(21) Appl. No.: 19/480,653

(22) PCT Filed: May 30, 2024
(86) PCT No.: PCT/US2024/031690

§ 371 Date: Oct. 31, 2025

Related U.S. Application Data
(60) Provisional application No. 63/505,829, filed on Jun. 2, 2023.

(51) Int. Cl. A61K 39/21; A61P 37/04; C07K 14/005

(52) U.S. Cl. CPC A61K 39/21; A61P 37/04; C07K 14/005

(57) ABSTRACT

Non-naturally occurring human immunodeficiency virus (HIV) antigens are provided, of the general formula X1-X2-X3-X4-X5-X6-X7-X8-X9-X10-X11 with each domain as defined herein, as are fusion proteins including such HIV antigens fused to a nanoparticle component, nanoparticles including such fusion proteins, nucleic acids encoding such HIV antigens and fusion proteins, and methods for using such reagents for treating or limiting development of an HIV infection.

### SEQUENCE LISTING STATEMENT

A computer readable form of the Sequence Listing is filed with this application by electronic submission and is incorporated into this application by reference in its entirety. The Sequence Listing is contained in the file created on May 21, 2024 having the file name “23-0645-WO” and is 130,152 bytes in size.

### BACKGROUND

Developing new immunogens for Human immunodeficiency 1 (HIV-1) vaccines aiming at inducing broadly neutralizing antibodies (bNAbs) requires addressing multiple features that make HIV-1 envelope protein (hereafter Env)—the sole antibody neutralization target of HIV-1—one of the most complex viral fusion protein to work with (e.g. metastability of gp41, variable loops, hetero-oligomer, glycosylation, sequence diversity, etc.) Off-target responses are the primary responses to Env immunogen, limiting the potential efficacy of such immunogens in repeated vaccination schedules that use similar immunogens at each immunization. In addition, monomeric Env immunogen such as engineered gp120 core and outer domain (eOD-GT) proteins—even presented on nanoparticles—lack antigenic context (i.e. adjacent protomer(s), glycans) which trimeric immunogens provide. Improved Env immunogens are needed.

### SUMMARY

In a first aspect, the disclosure provides non-naturally occurring HIV antigens, comprising the general formula X1-X2-X3-X4-X5-X6-X7-X8-X9-X10-X11, wherein:
X1 comprises an amino acid sequence at least 50%, 55%, 60%, 65%, 70%, 75%, 80%, 85%, 90%, 92%, 94%, 96%, 98%, or 100% identical to the amino acid sequence of (LTPL)CVTLQCTNVINNITDDMRGELKNCSFNMTTELRDKRQKVHALFYKLDIVPIN ENQNTTYRLINCNTA (SEQ ID NO:1), wherein residues in parentheses are optional and may be present or may be deleted;
X2 comprises an amino acid sequence at least 50%, 55%, 60%, 65%, 70%, 75%, 80%, 85%, 90%, 92%, 94%, 96%, 98%, or 100% identical to the amino acid sequence: QIVQWPPDVSLEPEPIYYCAP(P/C)(C/G)Y (SEQ ID NO:2), wherein the highlighted C residue cannot be substituted;
X3 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:3, or (SEQ ID NO:72);
X4 comprises an amino acid sequence at least 50%, 60%, 70%, 80%, 90%, 93%, or 100% identical to the amino acid sequence of SEQ ID NO:5;
X5 comprises an amino acid sequence at least 50%, 60%, 70%, 80%, 90%, 93%, or 100% identical to the amino acid sequence of SEQ ID NO:6;
X6 comprises an amino acid sequence at least 50%, 60%, 70%, 80%, 90%, 93%, or 100% identical to the amino acid sequence of SEQ ID NO:7;
X7 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:8;
X8 comprises an amino acid sequence of at least 3 contiguous amino acids identical to the amino acid sequence of SEQ ID NO:9, or at least 4 or 5, or all 6 contiguous amino acids identical to the amino acid sequence SEQ ID NO:9;
X9 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:10;
X10 comprises an amino acid sequence at least 50%, 60%, 70%, 80%, 90%, 93%, or 100% identical to the amino acid sequence of SEQ ID NO: 11; and
X11 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 12.

In one embodiment, the general formula comprises X1-X2-X3-X4-X5-X6-X7-X8-X9-X10-X11-X12-X13, wherein X1-X11 are as defined above, and wherein X12 comprises an amino acid sequence at least 20%, 40%, 60%, 89%, or 100% identical to the amino acid sequence of SEQ ID NO: 15; and
X13 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:16.

In another embodiment, the antigen comprises an amino acid sequence at least 50%, 55%, 60%, 65%, 70%, 75%, 80%, 85%, 90%, 92%, 94%, 96%, 98%, or 100% identical to the amino acid sequence the amino acid sequence selected from the group consisting of SEQ ID NO: 20-27 and 73-74.

In second aspect, the disclosure provides non-naturally occurring HIV antigens, comprising the general formula X1-X2-X3-X4-X5-X6-X7-X8-X9-X10-X11, wherein:
X1 comprises an amino acid sequence at least 50%, 55%, 60%, 65%, 70%, 75%, 80%, 85%, 90%, 92%, 94%, 96%, 98%, or 100% identical to the amino acid sequence of (LTPL)CVTLQCTNVINNITDDMRGELKNCSFNMTTELRDKRQKVHALFYKLDIVPIN ENQNTTYRLINCNTA (SEQ ID NO:1), or (LTPL)CVTLQCTNVSNNITDDMRGELKNCSFNMTTELRDKKQKVYSLFYRLDVVQI NENQGNRINNTNKEYRLINCNTT (SEQ ID NO:28), wherein residues in parentheses are optional and may be present or may be deleted;
X2 comprises an amino acid sequence at least 50%, 55%, 60%, 65%, 70%, 75%, 80%, 85%, 90%, 92%, 94%, 96%, 98%, or 100% identical to the amino acid sequence: SI(K/C)QW(P/C)PDVSLEPEPLYLCAP(K/C)GY (SEQ ID NO:29), wherein the highlighted C residue cannot be substituted;
X3 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:3 or SEQ ID NO:72;
X4 comprises an amino acid sequence at least 50%, 60%, 70%, 80%, 90%, 93%, or 100% identical to the amino acid sequence of SEQ ID NO:4 or 5;
X5 comprises an amino acid sequence at least 50%, 60%, 70%, 80%, 90%, 93%, or 100% identical to the amino acid sequence of SEQ ID NO:6;
X6 comprises an amino acid sequence at least 50%, 60%, 70%, 80%, 90%, 93%, or 100% identical to the amino acid sequence of SEQ ID NO:30;
X7 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:8;
X8 comprises an amino acid sequence of at least 3 contiguous amino acids identical to the amino acid sequence of SEQ ID NO:31, or at least 4 or all 5 contiguous amino acids identical to the amino acid sequence of SEQ ID NO:31;
X9 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:32;
X10 comprises an amino acid sequence at least 50%, 60%, 70%, 80%, 90%, 93%, or 100% identical to the amino acid sequence of SEQ ID NO: 11; and
X11 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:33.

In one embodiment of this second aspect, the general formula comprises X1-X2-X3-X4-X5-X6-X7-X8-X9-X10-X11-X12-X13, wherein X1-X11 are as defined above, and wherein
X12 comprises an amino acid sequence at least 50% or 100% identical to the amino acid sequence: (G/C)G(S/C)G (SEQ ID NO: 99); and
X13 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:37.

In a further embodiment, the antigen comprises an amino acid sequence at least 50%, 55%, 60%, 65%, 70%, 75%, 80%, 85%, 90%, 92%, 94%, 96%, 98%, or 100% identical to the amino acid sequence selected from the group consisting of SEQ ID NO:41-49 and 75-77.

In another aspect, the disclosure provides fusion proteins, comprising:
(a) the non-naturally occurring HIV antigen of any embodiment of the first or second aspects of the disclosure; and
(b) a protein nanoparticle component.

In one embodiment, the protein nanoparticle component is C-terminal to the non-naturally occurring HIV antigen. In another embodiment, the protein nanoparticle component comprises a trimer-forming polypeptide. In a further embodiment, the protein nanoparticle component comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:51. In one embodiment, the fusion protein comprises an amino acid sequence at least 50%, 55%, 60%, 65%, 70%, 75%, 80%, 85%, 90%, 92%, 94%, 96%, 98%, or 100% identical to the amino acid sequence selected from the group consisting of SEQ ID NO: 52-68 and 78-95, wherein the N-terminal signal sequence of SEQ ID NO:50 (when present) is optional, and may be present, or may be deleted, or may be replaced with another signal sequence.

In another aspect, the disclosure provides nanoparticles comprising multiple copies of the fusion protein of embodiment disclosed herein. In one embodiment, the nanoparticle comprises
(a) a plurality of first assemblies, each first assembly comprising a plurality of identical first proteins comprising an amino acid sequence at least 80%, 85%, 90%, 91% 92%, 93%, 94%, 95%, 96%, 97%, 98%, 99%, or 100% identical to the amino acid sequence of SEQ ID NO:69-71, where the N-terminal MG residues are optional and may be present or absent, and
(b) a plurality of second assemblies, each second assembly comprising a plurality of fusion proteins according to any embodiment herein;
wherein the plurality of first assemblies non-covalently interact with the plurality of second assemblies to form the nanoparticle; and
wherein the nanoparticle displays on its surface an immunogenic portion of non-naturally occurring HIV antigen.

The disclosure also provides nucleic acids encoding the non-naturally occurring HIV antigen or fusion protein of any aspect or embodiment herein. In one embodiment, the nucleic acid comprises RNA. In a further embodiment, the RNA comprises mRNA. In one embodiment, the RNA comprises nucleoside-modified RNA, including but not limited to NI-methylpseudouridine-5′-triphosphate containing RNA. In another embodiment, the RNA comprises self-amplifying mRNA. In a further embodiment, the nucleic acid encodes a poly A tail (DNA) or comprises a poly A tail (RNA). In one embodiment, the nucleic acid encodes a 5′ UTR and/or a 3′ UTR (DNA) or comprises a 5′ UTR and/or a 3′ UTR (RNA).

The disclosure also provides expression vectors comprising the nucleic acid of any embodiment operatively linked to a suitable control element, such as a promoter, and host cells comprising the non-naturally occurring HIV antigen, fusion protein, nanoparticle, nucleic acid or expression vector of any embodiment herein. The disclosure further provides pharmaceutical compositions or vaccines, comprising the non-naturally occurring HIV antigen, fusion protein, nanoparticle, nucleic acid, expression vector, or host cell of any embodiment herein and a pharmaceutically acceptable carrier. In one embodiment, the pharmaceutical composition or vaccine may further comprise an adjuvant.

The disclosure also provides methods to treat or limit development of an HIV infection, comprising administering to a subject in need thereof an amount effective to treat or limit development of the infection of the non-naturally occurring HIV antigen, fusion protein, nanoparticle, nucleic acid, expression vector, host cell, vaccine, and/or pharmaceutical composition of any embodiment herein.

### DESCRIPTION OF THE FIGURES

FIG. 1. Sequence alignment of a trimeric platform design. The amino acid sequences of the YA_024_gtapex design (SEQ ID NO: 100), GT1.2 v8.1 NxT (SEQ ID NO: 101) and HXB2 (SEQ ID NO: 102) reference strain are aligned respecting the de novo design gp120 permutation. Amino acid numbering is indicated in two ways: top, numbering of YA 024 gtapex starting after the signal peptide; bottom, corresponding HXB2 reference sequence numbering system. Secondary structure corresponding to the pre-fusion state of Env are indicated as well as sub-domains and domains. A key is provided for annotation of the designed sequence.

FIG. 2. Schematic of the design process to create de novo helix bundles. Helices were propagated from 153 dn5b N-termini using oligomeric hallucination with various seed sequences. De novo helix bundle+153 dn5b were then docked to the permutated relooped gp120 subunits.

FIG. 3. Examples of designs and their AlphaFold2™ predicted structures. Five designs are dipected. Four designs are shown as overlays of the design pdb and the predicted fold and one model (bottom right panel) is depicted. The top panels highlight the need for additional disulfide bonds after ProteinMPNN™ optimization in order to obtain folded trimers. The root mean square deviation (RMSD) to the design and to gp120 are indicated for each design, the pAE and ipTM scores are also indicated. The location of the disulfide bonds is indicated with either zoom-in images or with circles.

FIG. 4. The addition of disulfide bond(s) improves AlphaFold2™ predictions, AlphaFold2™ scores (i.e. pAE, pLDDT, ipTM) and RMSD to design backbones (‘non-MPNN design’) are plotted. The ‘MPNN+DS’ data points represent constructs derived from ‘MPNN-no DS’ parent constructs in which DS were added. DS, disulfide bond.

FIG. 5. Designs bind to PGT145 quaternary specific bNAb. Ni-NTA ELISA data showing binding curves for 4 anti-Env mAbs (2G12, PGT145, gl-PGV19 and VRC01).

FIG. 6. Disulfide bond(s) improve and/or modulate binding to PGT145. ELISA data are shown with 4 mAbs tested (2G12, PGT145, gl-PGV19 and VRC01). The top panels show YA_016 parent designs (germline apex and mature apex) and derived designs (YA_020_matureapex, YA_024_gtapex, YA_023_gtapex) which include disulfide bond(s) (DS) and bound to PGT145. The bottom panels show YA_036 parent designs as above and the derived designs containing DS. The intrachain designed DS are given with cysteine position numbers are indicated. When the designed DS were designed as inter-chain DS, it is indicated in brackets.

FIG. 7. SEC traces on Superose™ 6 Increase 5/150 GL confirm dominant trimer species after IMAC purification.

FIG. 8. ELISA mAbs panel demonstrate absence of gp41 and native-like features. Designs were probed for anti-Env mAbs binding. P2401: GT1.1 v8.1 CD4bs KO His. P2406: GT1.2 v8.1 His.

FIG. 9. Negative stain-electron microscopy reveals propeller shaped trimers. Samples from fraction 2 of the Superdex™ 200 Increase 5/150 GL purified protein were used analysed. Relative levels of binding to PGT145, PGDM1400 and VRC01 in ELISA are indicated.

FIG. 10. ELISA binding data from Superose™ 6 Increase 10/300 GL purified trimers (1/3).

FIG. 11. ELISA binding data from Superose™ 6 Increase 10/300 GL purified trimers (2/3).

FIG. 12. ELISA binding data from Superose™ 6 Increase 10/300 GL purified trimers (3/3).

FIG. 13. Weak affinity interaction mediates aggregation. (A) SEC traces of concentrated YA_016 series designs loaded onto a Superose™ 6 Increase 10/300 GL column showing aggregates and trimer peaks. (B) Lower amount (1.4 mg in 500 μL) of trimer YA_038 gtapex (38G) loaded onto a Superose™ 6 Increase 10/300 GL with or without addition of 20 mM EDTA at room temperature 10 min prior to injection 1.4 mg of in 500 μL without EDTA demonstrate that aggregate formation is dramatically reduced compared to 8.6 mg trimer loaded onto the column. (C) Collecting IMAC eluate into EDTA plus L-Arginine solution prevents aggregate formation. (D) SEC races of 36G, 37G and 39M designs after IMAC purification with elution into EDTA plus L-Arginine.

FIG. 14. Reducing SDS-PAGE of gp41-free trimer designs. 1-2 μg of protein were boiled for 5 min at 95° C. in SDS running buffer plus 10 mM DTT. Design names shortened to the design number plus G (for ‘gtapex’) or M (for ‘matureapex).

FIG. 15. gp41-free designs are highly thermostable. SYPRO Orange™ thermostability assay curves. Measurements were acquired on an Uncle™ device from Unchained Labs. Melting temperatures are indicated on the right end side of the figure. BG505 GT1.2 v8.1: control protein (stable Env SOSIP trimer). Mean of n≥2 (duplicate for control protein, triplicate for gp41-free trimer designs) with SDs indicated as shaded areas.

FIG. 16. Melting curves and aggregation profiles. Tryptophan fluorescence measurement reported as the barycentric mean (BCM) for designs from the YA_016 series (A) and the YA_036 series (B). Aggregation profile as assessed by static light scattering (SLS) at 473 nm for designs from the YA_016 series (C) and the YA_036 series (D). Mean of triplicate with SDs indicated as shaded areas.

FIG. 17. High thermostability of gp41-free designs after several freeze/thaw cycles. SYPRO Orange™ assay curves as measured using Uncle™ (Unchained Labs). YA_020 matureapex (20M), YA_023G_gtapex (23G), YA_036_gtapex (36G), YA_036_matureapex (36M), YA_038_gtapex (38G) and YA_038_matureapex (38M) designs were assessed for melting temperature after 14 days at +4° C. and after 1, 2 and 5 freeze/thaw (F/T) cycles. Mean of triplicate with SDs indicated as shaded areas.

FIG. 18. ELISA binding patterns remain unchanged after several freeze/thaw cycles. ELISA mAbs panel demonstrate the preserved closed native-like binding features after 14 days at +4° C. and after 1, 2 and 5 freeze/thaw (F/T) cycles. Design names shortened to the design number plus G (for ‘gtapex’) or M (for ‘matureapex).

FIG. 19. YA_038 designs display closed pre-fusion native-like trimer conformation by negative stain-electron microscopy. Negative strain-electron microscopy 2D-class averages of YA_038_matureapex and YA_038_gtapex in complex with VRC01 Fab. Relative levels of binding to PGT145, PGDM1400 and VRC01 in ELISA are given. YA_038_gtapex appeared to have well-ordered trimers and particles.

FIG. 20. Rigidly presented gp41-free Env designs on 153_dn5 nanoparticles. (A) SEC traces of particles following a 1:1 molar assembly of 153_dn5A.2 component with 153 dn5b component displaying 4 different gp41-free Env designs. (B) DLS measurement of 153_dn5 nanoparticles presenting 5 gp41-free Env designs and demonstrating monodispersed nanoparticles. (C) Negative strain-electron microscopy micrograph of YA_038_gtapex 153_dn5 particles. (D) Negative stain-electron microscopy 2D-class averages of YA_038_gtapex 153_dn5 nanoparticles showing well-ordered and rigidly attached trimeric gp120 subunits.

FIG. 21. Reducing SDS-PAGE of 153_dn5 particles. 0.4 to 2 μg of 153_dn5 particles were boiled for 5 min at 95° C. in SDS running buffer plus 10 mM DTT. Design names shortened to the design number plus G (for ‘gtapex’) or M (for ‘matureapex).

FIG. 22. In vitro activation of BCR engineered Ramos B cells. The de novo designs are able to activate Ramos B-cell that express anti-Env BCRs, including germline VRC26 (GL-VRC26) targeting the VIV2 apex. Negative control SARS-COV-2 Spike trimer does not induce responses as expected.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Any N-terminal methionine residues in polypeptides or polypeptide domains disclosed herein are optional and may be present or may be deleted.

The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

Disclosed herein are non-naturally occurring HIV antigens, and fusion proteins and nanoparticles comprising the fusion proteins, that exclude gp41 from HIV-1 Env immunogens while retaining closed pre-fusion native-like features, thus having the potential to limit off-target responses while focusing immune responses on gp120 epitopes of interest presented in the native trimer geometrical space.

In a first aspect, the disclosure provides non-naturally occurring HIV antigens, comprising the general formula X1-X2-X3-X4-X5-X6-X7-X8-X9-X10-X11, wherein:
X1 comprises an amino acid sequence at least 50%, 55%, 60%, 65%, 70%, 75%, 80%, 85%, 90%, 92%, 94%, 96%, 98%, or 100% identical to the amino acid sequence: (LTPL)CVTLQCTNVINNITDDMRGELKNCSFNMTTELRDKRQKVHALFYKLDIVPIN ENQNTTYRLINCNTA (SEQ ID NO:1), wherein residues in parentheses are optional and may be present or may be deleted;
X2 comprises an amino acid sequence at least 50%, 55%, 60%, 65%, 70%, 75%, 80%, 85%, 90%, 92%, 94%, 96%, 98%, or 100% identical to the amino acid sequence: QIVQWPPDVSLEPEPIYYCAP(P/C)(C/G)Y (SEQ ID NO:2), wherein the highlighted C residue cannot be substituted;
X3 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 3)
AILKCKDKKFNGTGPCTNVTTVQCTHGIKPVVSTQLLLNGTLAEE
EVMIRSEDIRDNAKNILVQFNTTVQINCTRPNNNTRKSIRIGPGQ
WFYATGDIIGDIRQAHCNVTKATWNETLGKVVKQLRKHFGNNTII
RFANSTGGDLEVTTHSFNCGGEFFYCDTSGLF,
or
(SEQ ID NO: 72)
AILKCKDKKFNGTGPCTNVTTVQCTHGIKPVVSTQLLLNGTLAEE
EVMIRSKDIRNAKNILVQFNTTVQINCTRPNNNTRKSIRIGPGQW
FYATGDIIGDIRQAHCNVTKATWNETLGKVVKQLRKHFGNNTIIR
FANSTGGDLEVTTHSFNCGGEFFYCDTSGLF;

X4 comprises an amino acid sequence at least 50%, 60%, 70%, 80%, 90%, 93%, or 100% identical to the amino acid sequence: NTTDDSNSTSDDNGT (SEQ ID NO:4) or NSTWISNTTVQGSNSTGSNDT (SEQ ID NO:5);
X5 comprises an amino acid sequence at least 50%, 60%, 70%, 80%, 90%, 93%, or 100% identical to the amino acid sequence: ITLPCRIKQIINMWQR (SEQ ID NO:6);
X6 comprises an amino acid sequence at least 50%, 60%, 70%, 80%, 90%, 93%, or 100% identical to the amino acid sequence: EGQYVYLPPVE (SEQ ID NO:7);
X7 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 8)
GVIRCVSNITGLILTRDGGSTDSTTETFRPSGGDMRDNWRSELYKYKVVK;

X8 comprises an amino acid sequence of at least 3 contiguous amino acids identical to the amino acid sequence: KVGGEG (SEQ ID NO:9), or at least 4 or 5, or all 6 contiguous amino acids identical to the amino acid sequence KVGGEG (SEQ ID NO:9);
X9 comprises an amino acid sequence at least 50%, 55%, 60%, 65%, 70%, 75%, 80%, 85%, 90%, 92%, 94%, 96%, 98%, or 100% identical to the amino acid sequence: NWEDASVKLFKACPPTVSPGKTGNYFTGPECPLVP (SEQ ID NO:10);
X10 comprises an amino acid sequence at least 50%, 60%, 70%, 80%, 90%, 93%, or 100% identical to the amino acid sequence: PNPQEIHLENVTEEFNMWKNN (SEQ ID NO: 11); and
X11 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 12)
MVDQVYEDVEYRFRLALG(D/C)AL.

As disclosed in the examples, the antigens of this aspect of the disclosure preserve the trimeric geometry of Env gp120 subunits in a closed pre-fusion native-like fashion mimicking the most advanced stabilized Env trimers while removing non-neutralizing immunodominant epitopes and unstable regions of Env. As such, the antigens can be used, for example, in HIV vaccines. In all embodiments herein, domains X1, X2, X4, X6, X8, X9, and X11 are engineered to preserves the trimeric geometry of Env gp120 subunits in a closed pre-fusion native-like fashion mimicking the most advanced stabilized Env trimers while removing non-neutralizing immunodominant epitopes and unstable regions of Env. Details of the engineering are provided in the examples that follow. Domains X3, X5, X7, and X10 domains are exemplary sequences unmodified from a BG505 HIV-1 strain, and may vary widely in view of Env sequence differences between strains.

In one embodiment,

X1 comprises the amino acid sequence: (LTPL)CVTLQCTNVTNNITDDMRGELKNCSFNMTTELRDKRQKVHALFYKLDIVPIN ENQNTTYRLINCNTA (SEQ ID NO:1), wherein residues in parentheses are optional and may be present or may be deleted;
X2 comprises the amino acid sequence: QIVQWPPDVSLEPEPIYYCAP(P/C)(C/G)Y (SEQ ID NO:2), wherein the highlighted C residue cannot be substituted;
X4 comprises the amino acid sequence: NTTDDSNSTSDDNGT (SEQ ID NO:4) or NSTWISNTTVQGSNSTGSNDT (SEQ ID NO:5);
X6 comprises the amino acid sequence: EGQYVYLPPVE (SEQ ID NO:7);
X8 comprises the amino acid sequence: KVGGEG (SEQ ID NO:9);
X9 comprises the amino acid sequence: NWEDASVKLFKACPPTVSPGKTGNYFTGPECPLVP (SEQ ID NO: 10); and
X11 comprises the amino acid sequence: MVDQVYEDVEYRFRLALG(D/C)AL (SEQ ID NO:12).

In another embodiment,
X1 comprises the amino acid sequence: (LTPL)CVTLQCTNVINNITDDMRGELKNCSFNMTTELRDKRQKVHALFYKLDIVPIN ENQNTTYRLINCNTA (SEQ ID NO:1), wherein residues in parentheses are optional and may be present or may be deleted;
X2 comprises the amino acid sequence: QIVQWPPDVSLEPEPIYYCAP(P/C)(C/G)Y (SEQ ID NO:2), wherein the highlighted C residue cannot be substituted;
X3 comprises the amino acid sequence:

(SEQ ID NO: 3)
AILKCKDKKENGTGPCTNVTTVQCTHGIKPVVSTQLLLNGTLAEE
EVMIRSEDIRDNAKNILVQFNTTVQINCTRPNNNTRKSIRIGPGQ
WFYATGDIIGDIRQAHCNVTKATWNETLGKVVKQLRKHFGNNTII
RFANSTGGDLEVTTHSFNCGGEFFYCDTSGLF,
or
(SEQ ID NO: 72)
AILKCKDKKFNGTGPCTNVTTVQCTHGIKPVVSTQLLLNGTLAEE
EVMIRSKDIRNAKNILVQFNTTVQINCTRPNNNTRKSIRIGPGQW
FYATGDIIGDIRQAHCNVTKATWNETLGKVVKQLRKHFGNNTIIR
FANSTGGDLEVTTHSFNCGGEFFYCDTSGLF;

X4 comprises the amino acid sequence: NTTDDSNSTSDDNGT (SEQ ID NO:4) or NSTWISNTTVQGSNSTGSNDT (SEQ ID NO:5);
X5 comprises the amino acid sequence: ITLPCRIKQUINMWQR (SEQ ID NO:6);
X6 comprises the amino acid sequence: EGQYVYLPPVE (SEQ ID NO:7);
X7 comprises the amino acid sequence:

(SEQ ID NO: 8)
GVIRCVSNITGLILTRDGGSTDSTTETFRPSGGDMRDNWRS
ELYKYKVVK;

X8 comprises the amino acid sequence: KVGGEG (SEQ ID NO:9);
X9 comprises the amino acid sequence: NWEDASVKLFKACPPTVSPGKTGNYFTGPECPLVP (SEQ ID NO:10);
X10 comprises the amino acid sequence: PNPQEIHLENVTEEFNMWKNN (SEQ ID NO: 11); and
X11 comprises the amino acid sequence: MVDQVYEDVEYRFRLALG(D/C)AL (SEQ ID NO:12).

In one embodiment, X11 comprises the amino acid sequence: MVDQVYEDVEYRFRLALGCAL (SEQ ID NO: 13); in another embodiment, X11 comprises the amino acid sequence: MVDQVYEDVEYRFRLALGDAL (SEQ ID NO:14).

In another embodiment, the HIV antigen comprises the general formula X1-X2-X3-X4-X5-X6-X7-X8-X9-X10-X11-X12-X13, wherein X1-X11 are as defined above, and wherein
X12 comprises an amino acid sequence at least 20%, 40%, 60%, 89%, or 100% identical to the amino acid sequence: TGRAP (SEQ ID NO: 15); and
X13 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 16)
A(A/C)AEARRHHQRIEELERRQAEIE(G/C)RQAEAEREL.

The addition of domains X12 and X13 provides a helical bundle that facilitates integration of the antigens into plasma membranes or nanoparticles, such as the trimeric nanoparticles exemplified below.

In one embodiment,
X12 comprises the amino acid sequence: TGRAP (SEQ ID NO:15); and
X13 comprises the amino acid sequence: A(A/C)AEARRHHQRIEELERRQAEIE(G/C)RQAEAEREL (SEQ ID NO: 16). In another embodiment, X13 comprises the amino acid sequence selected from the group consisting of: AAAEARRHHQRIEELERRQAEIEGRQAEAEREL (SEQ ID NO:17), or AAAEARRHHQRIEELERRQAEIECRQAEAEREL (SEQ ID NO:18), or ACAEARRHHQRIEELERRQAEIECRQAEAEREL (SEQ ID NO: 19). In a specific embodiment, X13 comprises the amino acid sequence of SEQ ID NO: 19.

In other embodiments, the HIV antigen comprises an amino acid sequence at least 50%, 55%, 60%, 65%, 70%, 75%, 80%, 85%, 90%, 92%, 94%, 96%, 98%, or 100% identical to the amino acid sequence the amino acid sequence selected from the group consisting of SEQ ID NO:20-27 and 73-74. The sequences of SEQ ID NO:20-27 and 73-74 are shown in Table 1. In one embodiment, the HIV antigen comprises an amino acid sequence at least 75% identical to the amino acid sequence the amino acid sequence selected from the group consisting of SEQ ID NO:20-27 and 73-74. In another embodiment, the HIV antigen comprises an amino acid sequence at least 90% identical to the amino acid sequence the amino acid sequence selected from the group consisting of SEQ ID NO:20-27 and 73-74.

TABLE 1
YA_016_gtapex
LTPLCVTLQCTNVINNITDDMRGELKNCSENMTTELRDKRQKVHALFYKLDIVPINENQNTTYRLI
NCNTAQIVQWPPDVSLEPEPIYYCAPPGYAILKCKDKKENGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSENCGGEFFYCDTSGLENT
TDDSNSTSDDNGTITLPCRIKQIINMWQREGQYVYLPPVEGVIRCVSNITGLILTRDGGSTDSTTE
TFRPSGGDMRDNWRSELYKYKVVKKVGGEGNWEDASVKLFKACPPTVSPGKTGNYFTGPECPLVPP
NPQEIHLENVTEEFNMWKNNMVDQVYEDVEYRERLALGDAL (SEQ ID NO: 20)
YA_016_matureapex
LTPLCVTLQCTNVSNNITDDMRGELKNCSENMTTELRDKKQKVYSLFYRLDVVQINENQGNRINNT
NKEYRLINCNTTQIVQWPPDVSLEPEPIYYCAPPGYAILKCKDKKENGTGPCTNVTTVQCTHGIKP
VVSTQLLINGTLAEEEVMIRSEDIRDNAKNILVQENTTVQINCTRPNNNTRKSIRIGPGQWFYATG
DIIGDIRQAHCNVTKATWNETLGKVVKQLRKHFGNNTIIRFANSTGGDLEVTTHSFNCGGEFFYCD
TSGLFNTTDDSNSTSDDNGTITLPCRIKQIINMWQREGQYVYLPPVEGVIRCVSNITGLILTRDGG
STDSTTETFRPSGGDMRDNWRSELYKYKVVKKVGGEGNWEDASVKLFKACPPTVSPGKTGNYFTGP
ECPLVPPNPQEIHLENVTEEFNMWKNNMVDQVYEDVEYRERLALGDAL (SEQ ID NO: 21)
YA_020_gtapex
LTPLCVTLQCTNVINNITDDMRGELKNCSENMTTELRDKRQKVHALFYKLDIVPINENQNTTYRLI
NCNTAQIVQWPPDVSLEPEPIYYCAPCGYAILKCKDKKENGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLENT
TDDSNSTSDDNGTITLPCRIKQIINMWQREGQYVYLPPVEGVIRCVSNITGLILTRDGGSTDSTTE
TFRPSGGDMRDNWRSELYKYKVVKKVGGEGNWEDASVKLFKACPPTVSPGKTGNYFTGPECPLVPP
NPQEIHLENVTEEFNMWKNNMVDQVYEDVEYRERLALGDAL (SEQ ID NO: 22)
YA_020_matureapex
LTPLCVTLQCTNVSNNITDDMRGELKNCSENMTTELRDKKQKVYSLFYRLDVVQINENQGNRINNT
NKEYRLINCNTTQIVQWPPDVSLEPEPIYYCAPCGYAILKCKDKKENGTGPCTNVTTVQCTHGIKP
VVSTQLLINGTLAEEEVMIRSEDIRDNAKNILVQFNTTVQINCTRPNNNTRKSIRIGPGQWFYATG
DIIGDIRQAHCNVTKATWNETLGKVVKQLRKHEGNNTIIRFANSTGGDLEVTTHSENCGGEFFYCD
TSGLFNTTDDSNSTSDDNGTITLPCRIKQIINMWQREGQYVYLPPVEGVIRCVSNITGLILTRDGG
STDSTTETFRPSGGDMRDNWRSELYKYKVVKKVGGEGNWEDASVKLFKACPPTVSPGKTGNYFTGP
ECPLVPPNPQEIHLENVTEEFNMWKNNMVDQVYEDVEYRERLALGDAL (SEQ ID NO: 23)
YA_023_gtapex
LTPLCVTLQCTNVINNITDDMRGELKNCSENMTTELRDKRQKVHALFYKLDIVPINENQNTTYRLI
NCNTAQIVQWPPDVSLEPEPIYYCAPCGYAILKCKDKKFNGTGPCTNVTTVQCTHGIKPVVSTQLL
LNGTLAEEEVMIRSEDIRDNAKNILVQENTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLENT
TDDSNSTSDDNGTITLPCRIKQIINMWQREGQYVYLPPVEGVIRCVSNITGLILTRDGGSTDSTTE
TFRPSGGDMRDNWRSELYKYKVVKKVGGEGNWEDASVKLFKACPPTVSPGKTGNYFTGPECPLVPP
NPQEIHLENVTEEENMWKNNMVDQVYEDVEYRFRLALGCAL (SEQ ID NO: 24)
YA_023_matureapex
LTPLCVTLQCINVSNNITDDMRGELKNCSENMTTELRDKKQKVYSLFYRLDVVQINENQGNRINNT
NKEYRLINCNTTQIVQWPPDVSLEPEPIYYCAPCGYAILKCKDKKFNGTGPCTNVTTVQCTHGIKP
VVSTQLLINGTLAEEEVMIRSEDIRDNAKNILVQENTTVQINCTRPNNNTRKSIRIGPGQWFYATG
DIIGDIRQAHCNVTKATWNETLGKVVKQLRKHFGNNTIIRFANSTGGDLEVTTHSENCGGEFFYCD
TSGLFNTTDDSNSTSDDNGTITLPCRIKQIINMWQREGQYVYLPPVEGVIRCVSNITGLILTRDGG
STDSTTETFRPSGGDMRDNWRSELYKYKVVKKVGGEGNWEDASVKLFKACPPTVSPGKTGNYFTGP
ECPLVPPNPQEIHLENVTEEENMWKNNMVDQVYEDVEYRERLALGCAL (SEQ ID NO: 25)
YA_024_gtapex
LTPLCVTLQCINVINNITDDMRGELKNCSENMTTELRDKRQKVHALFYKLDIVPINENQNTTYRLI
NCNTAQIVQWPPDVSLEPEPIYYCAPPCYAILKCKDKKENGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSENCGGEFFYCDTSGLENT
TDDSNSTSDDNGTITLPCRIKQIINMWQREGQYVYLPPVEGVIRCVSNITGLILTRDGGSTDSTTE
TERPSGGDMRDNWRSELYKYKVVKKVGGEGNWEDASVKLFKACPPTVSPGKTGNYFTGPECPLVPP
NPQEIHLENVTEEENMWKNNMVDQVYEDVEYRERLALGCAL (SEQ ID NO: 26)
YA_024_matureapex
LTPLCVTLQCTNVSNNITDDMRGELKNCSENMTTELRDKKQKVYSLFYRLDVVQINENQGNRINNT
NKEYRLINCNTTQIVQWPPDVSLEPEPIYYCAPPCYAILKCKDKKENGTGPCTNVTTVQCTHGIKP
VVSTQLLINGTLAEEEVMIRSEDIRDNAKNILVQFNTTVQINCTRPNNNTRKSIRIGPGQWFYATG
DIIGDIRQAHCNVTKATWNETLGKVVKQLRKHEGNNTIIRFANSTGGDLEVTTHSENCGGEFFYCD
TSGLFNTTDDSNSTSDDNGTITLPCRIKQIINMWQREGQYVYLPPVEGVIRCVSNITGLILTRDGG
STDSTTETFRPSGGDMRDNWRSELYKYKVVKKVGGEGNWEDASVKLFKACPPTVSPGKTGNYFTGP
ECPLVPPNPQEIHLENVTEEFNMWKNNMVDQVYEDVEYRERLALGCAL (SEQ ID NO: 27)
YA_020_matureapex gt1.1
LTPLCVTLQCTNVSNNITDDMRGELKNCSFNMTTELRDKKQKVYSLFYRLDVVQINENQGNRINNT
NKEYRLINCNTTQIVQWPPDVSLEPEPIYYCAPCGYAILKCKDKKENGTGPCTNVTTVQCTHGIKP
VVSTQLLINGTLAEEEVMIRSKDIRNNAKNILVQENTTVQINCTRPNNNTRKSIRIGPGQWFYATG
DIIGDIRQAHCNVTKATWNETLGKVVKQLRKHFGNNTIIRFANSTGGDLEVTTHSENCGGEFFYCD
TSGLENSTWISNTTVQGSNSTGSNDTITLPCRIKQIINMWQREGQYVYLPPVEGVIRCVSNITGLI
LTRDGGSTDSTTETFRPSGGDMRDNWRSELYKYKVVKKVGGEGNWEDASVKLFKACPPTVSPGKTG
NYFTGPECPLVPPNPQEIHLENVTEEFNMWKNNMVDQVYEDVEYRFRLALGDAL
(SEQ ID NO: 73)
YA_024_gtapex gt1.1
ITPLCVTLQCTNVINNITDDMRGELKNCSFNMTTELRDKRQKVHALFYKLDIVPINENQNTTYRLI
NCNTAQIVQWPPDVSLEPEPIYYCAPPCYAILKCKDKKENGTGPCTNVTTVQCTHGIKPVVSTQLL
LNGTLAEEEVMIRSKDIRNNAKNILVQFNTTVQINCTRPNNNTRKSIRIGPGQWFYATGDIIGDIR
QAHCNVTKATWNETLGKVVKQLRKHEGNNTIIRFANSTGGDLEVTTHSENCGGEFFYCDTSGLENS
TWISNTTVQGSNSTGSNDTITLPCRIKQIINMWQREGQYVYLPPVEGVIRCVSNITGLILTRDGGS
TDSTTETFRPSGGDMRDNWRSELYKYKVVKKVGGEGNWEDASVKLFKACPPTVSPGKTGNYFTGPE
CPLVPPNPQEIHLENVTEEFNMWKNNMVDQVYEDVEYRERLALGCAL (SEQ ID NO: 74)

In a second aspect, the disclosure provides non-naturally occurring HIV antigen, comprising the general formula X1-X2-X3-X4-X5-X6-X7-X8-X9-X10-X11, wherein:
X1 comprises an amino acid sequence at least 50%, 55%, 60%, 65%, 70%, 75%, 80%, 85%, 90%, 92%, 94%, 96%, 98%, or 100% identical to the amino acid sequence: (LTPL)CVTLQCTNVTNNITDDMRGELKNCSFNMTTELRDKRQKVHALFYKLDIVPIN ENQNTTYRLINCNTA (SEQ ID NO:1), or (LTPL)CVTLQCTNVSNNITDDMRGELKNCSFNMTTELRDKKQKVYSLFYRLDVVQI NENQGNRINNINKEYRLINCNTT (SEQ ID NO:28), wherein residues in parentheses are optional and may be present or may be deleted;
X2 comprises an amino acid sequence at least 50%, 55%, 60%, 65%, 70%, 75%, 80%, 85%, 90%, 92%, 94%, 96%, 98%, or 100% identical to the amino acid sequence: SI(K/C)QW(P/C)PDVSLEPEPLYLCAP(K/C)GY (SEQ ID NO:29), wherein the highlighted C residue cannot be substituted;
X3 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 3)
AILKCKDKKFNGTGPCTNVTTVQCTHGIKPVVSTQLLLNGTLAEEEVMI
RSEDIRDNAKNILVQFNTTVQINCTRPNNNTRKSIRIGPGQWFYATGDI
IGDIRQAHCNVTKATWNETLGKVVKQLRKHFGNNTIIRFANSTGGDLEV
TTHSFNCGGEFFYCDTSGLF,
or
(SEQ ID NO: 72)
AILKCKDKKFNGTGPCTNVTTVQCTHGIKPVVSTQLLLNGTLAEEEVMI
RSKDIRNAKNILVQFNTTVQINCTRPNNNTRKSIRIGPGQWFYATGDII
GDIRQAHCNVTKATWNETLGKVVKQLRKHFGNNTIIRFANSTGGDLEVT
THSFNCGGEFFYCDTSGLF;

X4 comprises an amino acid sequence at least 50%, 60%, 70%, 80%, 90%, 93%, or 100% identical to the amino acid sequence: NTTDDSNSTSDDNGT (SEQ ID NO:4) or NSTWISNTTVQGSNSTGSNDT (SEQ ID NO:5);
X5 comprises an amino acid sequence at least 50%, 60%, 70%, 80%, 90%, 93%, or 100% identical to the amino acid sequence: ITLPCRIKQIINMWQR (SEQ ID NO:6);
X6 comprises an amino acid sequence at least 50%, 60%, 70%, 80%, 90%, 93%, or 100% identical to the amino acid sequence: NGLYVYLPPVE (SEQ ID NO:30);
X7 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 8)
GVIRCVSNITGLILTRDGGSTDSTTETFRPSGGDMRDNWRSELYKYKVV
K;

X8 comprises an amino acid sequence of at least 3 contiguous amino acids identical to the amino acid sequence: LGGEG (SEQ ID NO:31), or at least 4 or all 5 contiguous amino acids identical to the amino acid sequence LGGEG (SEQ ID NO:31);
X9 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 32)
KWEDASVKLYPAAPPTVSPGKTGNWLTGDKVLPTP;

X10 comprises an amino acid sequence at least 50%, 60%, 70%, 80%, 90%, 93%, or 100% identical to the amino acid sequence: PNPQEIHLENVTEEFNMWKNN (SEQ ID NO: 11); and
X11 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 33)
MVDQLSEYVQEQF(D/C)LALAG(S/C)N.

Similar to the antigens of the first aspect, the antigens of this second aspect of the disclosure also preserve the trimeric geometry of Env gp120 subunits in a closed pre-fusion native-like fashion mimicking the most advanced stabilized Env trimers while removing non-neutralizing immunodominant epitopes and unstable regions of Env. As such, the antigens can be used, for example, in HIV vaccines.

In one embodiment,
X1 comprises the amino acid sequence: (LTPL)CVTLQCTNVTNNITDDMRGELKNCSFNMTTELRDKRQKVHALFYKLDIVPIN ENQNTTYRLINCNTA (SEQ ID NO:1); or (LTPL)CVTLQCTNVSNNITDDMRGELKNCSFNMTTELRDKKQKVYSLFYRLDVVQI NENQGNRTNNTNKEYRLINCNTT (SEQ ID NO:28), wherein residues in parentheses are optional and may be present or may be deleted
X2 comprises the amino acid sequence: SI(K/C)QW(P/C)PDVSLEPEPLYLCAP(K/C)GY (SEQ ID NO:29), wherein the highlighted C residue cannot be substituted;
X4 comprises the amino acid sequence: NTTDDSNSTSDDNGT (SEQ ID NO:4) or NSTWISNTTVQGSNSTGSNDT (SEQ ID NO:5);
X6 comprises the amino acid sequence: NGLYVYLPPVE (SEQ ID NO:30);
X8 comprises the amino acid sequence: LGGEG (SEQ ID NO:31);
X9 comprises the amino acid sequence: KWEDASVKLFKACPPTVSPGKTGNYFTGPECPLVP (SEQ ID NO:34); and
X11 comprises the amino acid sequence: MVDQLSEYVQEQF(D/C)LALAG (S/C) N (SEQ ID NO:33).

In another embodiment,
X1 comprises the amino acid sequence: (LTPL)CVTLQCTNVTNNITDDMRGELKNCSFNMTTELRDKRQKVHALFYKLDIVPIN ENQNTTYRLINCNTA (SEQ ID NO:1), wherein residues in parentheses are optional and may be present or may be deleted
X2 comprises the amino acid sequence: SI(K/C)QW(P/C)PDVSLEPEPLYLCAP(K/C)GY (SEQ ID NO:29), wherein the highlighted C residue cannot be substituted;
X3 comprises the amino acid sequence:

(SEQ ID NO: 3)
AILKCKDKKFNGTGPCTNVTTVQCTHGIKPVVSTQLLLNGTLAEEEVMI
RSEDIRDNAKNILVQFNTTVQINCTRPNNNTRKSIRIGPGQWFYATGDI
IGDIRQAHCNVTKATWNETLGKVVKQLRKHFGNNTIIRFANSTGGDLEV
TTHSFNCGGEFFYCDTSGLF,
or
(SEQ ID NO: 72)
AILKCKDKKFNGTGPCTNVTTVQCTHGIKPVVSTQLLLNGTLAEEEVMI
RSKDIRNAKNILVQFNTTVQINCTRPNNNTRKSIRIGPGQWFYATGDII
GDIRQAHCNVTKATWNETLGKVVKQLRKHFGNNTIIRFANSTGGDLEVT
THSFNCGGEFFYCDTSGLF;

X4 comprises the amino acid sequence: NTTDDSNSTSDDNGT (SEQ ID NO:4) or NSTWISNTTVQGSNSTGSNDT (SEQ ID NO:5);
X5 comprises the amino acid sequence: ITLPCRIKQIINMWQR (SEQ ID NO:6);
X6 comprises the amino acid sequence: NGLYVYLPPVE (SEQ ID NO:30);
X7 comprises the amino acid sequence:

(SEQ ID NO: 8)
GVIRCVSNITGLILTRDGGSTDSTTETFRPSGGDMRDNWRSELYKYKVV
K;

X8 comprises the amino acid sequence: LGGEG (SEQ ID NO:31);
X9 comprises the amino acid sequence:

(SEQ ID NO: 34)
KWEDASVKLFKACPPTVSPGKTGNYFTGPECPLVP;

X10 comprises the amino acid sequence: PNPQEIHLENVTEEFNMWKNN (SEQ ID NO: 11); and
X11 comprises the amino acid sequence: MVDQLSEYVQEQF(D/C)LALAG (S/C) N (SEQ ID NO:33).

In some embodiments, X11 comprises the amino acid sequence MVDQLSEYVQEQFDLALAGSCN (SEQ ID NO:35); in other embodiments, X11 comprises the amino acid sequence MVDQLSEYVQEQFCLALAGEN (SEQ ID NO:36).

In another embodiment, the HIV antigen comprises the general formula X1-X2-X3-X4-X5-X6-X7-X8-X9-X10-X11-X12-X13, wherein X1-X11 are as defined in claims 11-15, and wherein
X12 comprises an amino acid sequence at least 50% or 100% identical to the amino acid sequence: (G/C)G(S/C)G; and
X13 comprises an amino acid sequence at least 50%, 55%, 60%, 65%, 70%, 75%, 80%, 85%, 90%, 92%, 94%, 96%, 98%, or 100% identical to the amino acid sequence:

(SEQ ID NO: 37)
S(T/C)A(A/C)LLKALQATKARADANALE(I/C)AEIKERIRETERE
L.

In one embodiment,
X12 comprises the amino acid sequence: (G/C)G(S/C)G; and
X13 comprises the amino acid sequence:

(SEQ ID NO: 37)
S(T/C)A(A/C)LLKALQATKARADANALE(I/C)AEIKERIRETERE
L.

In another embodiment, X13 comprises the amino acid sequence selected from the group consisting of SEQ ID NO:38-40.

(SEQ ID NO: 38)
STAALLKALQATKARADANALEIAEIKERIRETEREL,
or
(SEQ ID NO: 39)
SCACLLKALQATKARADANALEICEIKERIRETEREL,
or
(SEQ ID NO: 40)
STACLLKALQATKARADANALEIAEIKERIRETEREL.

In various embodiments, X12 may comprise the amino acid sequence GGSG (SEQ ID NO: 96), or CGCG (SEQ ID NO: 97), or CGSG (SEQ ID NO: 98), or GGSG (SEQ ID NO: 96).

In a further embodiment, the HIV antigen comprises an amino acid sequence at least 50%, 55%, 60%, 65%, 70%, 75%, 80%, 85%, 90%, 92%, 94%, 96%, 98%, or 100% identical to the amino acid sequence selected from the group consisting of SEQ ID NO:41-49 and 75-77. The amino acid sequences of SEQ ID NO:41-49 and 75-77 are provided in Table 2. In one embodiment, the HIV antigen comprises an amino acid sequence at least 75% identical to the amino acid sequence selected from the group consisting of SEQ ID NO:41-49 and 75-77. In another embodiment, the HIV antigen comprises an amino acid sequence at least 90% identical to the amino acid sequence selected from the group consisting of SEQ ID NO:41-49 and 75-77.

TABLE 2
YA_036_gtapex
LTPLCVTLQCTNVTNNITDDMRGELKNCSFNMTTELRDKRQKVHALFYKLDIVPINENQNTTYRLI
NCNTASIKQWPPDVSLEPEPLYLCAPKGYAILKCKDKKFNGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNT
TDDSNSTSDDNGTITLPCRIKQIINMWQRNGLYVYLPPVEGVIRCVSNITGLILTRDGGSTDSTTE
TFRPSGGDMRDNWRSELYKYKVVKILGGEGKWEDASVKLYPAAPPTVSPGKTGNWLTGDKVLPTPP
NPQEIHLENVTEEFNMWKNNMVDQLSEYVQEQFDLALAGSN (SEQ ID NO: 41)
YA_036_matureapex
LTPLCVTLQCTNVSNNITDDMRGELKNCSFNMTTELRDKKQKVYSLFYRLDVVQINENQGNRTNNT
NKEYRLINCNTTSIKQWPPDVSLEPEPLYLCAPKGYAILKCKDKKFNGTGPCTNVTTVQCTHGIKP
VVSTQLLLNGTLAEEEVMIRSEDIRDNAKNILVQFNTTVQINCTRPNNNTRKSIRIGPGQWFYATG
DIIGDIRQAHCNVTKATWNETLGKVVKQLRKHFGNNTIIRFANSTGGDLEVTTHSFNCGGEFFYCD
TSGLFNTTDDSNSTSDDNGTITLPCRIKQIINMWQRNGLYVYLPPVEGVIRCVSNITGLILTRDGG
STDSTTETFRPSGGDMRDNWRSELYKYKVVKILGGEGKWEDASVKLYPAAPPTVSPGKTGNWLTGD
KVLPTPPNPQEIHLENVTEEFNMWKNNMVDQLSEYVQEQFDLALAGSN (SEQ ID NO: 42)
YA_037_gtapex
LTPLCVTLQCTNVTNNITDDMRGELKNCSFNMTTELRDKRQKVHALFYKLDIVPINENQNTTYRLI
NCNTASICQWPPDVSLEPEPLYLCAPCGYAILKCKDKKFNGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNT
TDDSNSTSDDNGTITLPCRIKQIINMWQRNGLYVYLPPVEGVIRCVSNITGLILTRDGGSTDSTTE
TFRPSGGDMRDNWRSELYKYKVVKILGGEGKWEDASVKLYPAAPPTVSPGKTGNWLTGDKVLPTPP
NPQEIHLENVTEEFNMWKNNMVDQLSEYVQEQFCLALAGSN (SEQ ID NO: 43)
YA_037_matureapex
LTPLCVTLQCTNVSNNITDDMRGELKNCSFNMTTELRDKKQKVYSLFYRLDVVQINENQGNRTNNT
NKEYRLINCNTTSICQWPPDVSLEPEPLYLCAPCGYAILKCKDKKFNGTGPCTNVTTVQCTHGIKP
VVSTQLLINGTLAEEEVMIRSEDIRDNAKNILVQFNTTVQINCTRPNNNTRKSIRIGPGQWFYATG
DIIGDIRQAHCNVTKATWNETLGKVVKQLRKHFGNNTIIRFANSTGGDLEVTTHSFNCGGEFFYCD
TSGLFNTTDDSNSTSDDNGTITLPCRIKQIINMWQRNGLYVYLPPVEGVIRCVSNITGLILTRDGG
STDSTTETFRPSGGDMRDNWRSELYKYKVVKILGGEGKWEDASVKLYPAAPPTVSPGKTGNWLTGD
KVLPTPPNPQEIHLENVTEEFNMWKNNMVDQLSEYVQEQFCLALAGSN (SEQ ID NO: 44)
YA_038_gtapex
LTPLCVTLQCTNVTNNITDDMRGELKNCSFNMTTELRDKRQKVHALFYKLDIVPINENQNTTYRLI
NCNTASICQWPPDVSLEPEPLYLCAPKGYAILKCKDKKFNGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNT
TDDSNSTSDDNGTITLPCRIKQIINMWQRNGLYVYLPPVEGVIRCVSNITGLILTRDGGSTDSTTE
TFRPSGGDMRDNWRSELYKYKVVKILGGEGKWEDASVKLYPAAPPTVSPGKTGNWLTGDKVLPTPP
NPQEIHLENVTEEFNMWKNNMVDQLSEYVQEQFCLALAGSN (SEQ ID NO: 45)
YA_038_matureapex
LTPLCVTLQCTNVSNNITDDMRGELKNCSFNMTTELRDKKQKVYSLFYRLDVVQINENQGNRTNNT
NKEYRLINCNTTSICQWPPDVSLEPEPLYLCAPKGYAILKCKDKKFNGTGPCTNVTTVQCTHGIKP
VVSTQLLLNGTLAEEEVMIRSEDIRDNAKNILVQFNTTVQINCTRPNNNTRKSIRIGPGQWFYATG
DIIGDIRQAHCNVTKATWNETLGKVVKQLRKHFGNNTIIRFANSTGGDLEVTTHSFNCGGEFFYCD
TSGLFNTTDDSNSTSDDNGTITLPCRIKQIINMWQRNGLYVYLPPVEGVIRCVSNITGLILTRDGG
STDSTTETFRPSGGDMRDNWRSELYKYKVVKILGGEGKWEDASVKLYPAAPPTVSPGKTGNWLTGD
KVLPTPPNPQEIHLENVTEEFNMWKNNMVDQLSEYVQEQFCLALAGSN (SEQ ID NO: 46)
YA_038_gtapex_K401N
LTPLCVTLQCTNVTNNITDDMRGELKNCSFNMTTELRDKRQKVHALFYKLDIVPINENQNTTYRLI
NCNTASICQWPPDVSLEPEPLYLCAPKGYAILKCKDKKFNGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNT
TDDSNSTSDDNGTITLPCRIKQIINMWQRNGLYVYLPPVEGVIRCVSNITGLILTRDGGSTDSTTE
TFRPSGGDMRDNWRSELYKYKVVKILGGEGKWEDASVKLYPAAPPTVSPGNTGNWLTGDKVLPTPP
NPQEIHLENVTEEFNMWKNNMVDQLSEYVQEQFCLALAGSN (SEQ ID NO: 47)
YA_039_gtapex
LTPLCVTLQCTNVTNNITDDMRGELKNCSFNMTTELRDKRQKVHALFYKLDIVPINENQNTTYRLI
NCNTASIKQWCPDVSLEPEPLYLCAPKGYAILKCKDKKFNGTGPCTNVTTVQCTHGIKPVVSTQLL
LNGTLAEEEVMIRSEDIRDNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNT
TDDSNSTSDDNGTITLPCRIKQIINMWQRNGLYVYLPPVEGVIRCVSNITGLILTRDGGSTDSTTE
TFRPSGGDMRDNWRSELYKYKVVKILGGEGKWEDASVKLYPAAPPTVSPGKTGNWLTGDKVLPTPP
NPQEIHLENVTEEFNMWKNNMVDQLSEYVQEQFCLALAGCN (SEQ ID NO: 48)
YA_039_matureapex
LTPLCVTLQCTNVSNNITDDMRGELKNCSFNMTTELRDKKQKVYSLFYRLDVVQINENQGNRTNNT
NKEYRLINCNTTSIKQWCPDVSLEPEPLYLCAPKGYAILKCKDKKFNGTGPCTNVTTVQCTHGIKP
VVSTQLLLNGTLAEEEVMIRSEDIRDNAKNILVQFNTTVQINCTRPNNNTRKSIRIGPGQWFYATG
DIIGDIRQAHCNVTKATWNETLGKVVKQLRKHFGNNTIIRFANSTGGDLEVTTHSFNCGGEFFYCD
TSGLFNTTDDSNSTSDDNGTITLPCRIKQIINMWQRNGLYVYLPPVEGVIRCVSNITGLILTRDGG
STDSTTETFRPSGGDMRDNWRSELYKYKVVKILGGEGKWEDASVKLYPAAPPTVSPGKTGNWLTGD
KVLPTPPNPQEIHLENVTEEFNMWKNNMVDQLSEYVQEQFCLALAGCN (SEQ ID NO: 49)
YA_036_gtapex_gt1.1
LTPLCVTLQCTNVTNNITDDMRGELKNCSFNMTTELRDKRQKVHALFYKLDIVPINENQNTTYRLI
NCNTASIKQWPPDVSLEPEPLYLCAPKGYAILKCKDKKFNGTGPCTNVTTVQCTHGIKPVVSTQLL
INGTLAEEEVMIRSKDIRNNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNS
TWISNTTVQGSNSTGSNDTITLPCRIKQIINMWQRNGLYVYLPPVEGVIRCVSNITGLILTRDGGS
TDSTTETFRPSGGDMRDNWRSELYKYKVVKILGGEGKWEDASVKLYPAAPPTVSPGKTGNWLTGDK
VLPTPPNPQEIHLENVTEEFNMWKNNMVDQLSEYVQEQFDLALAGSN (SEQ ID NO: 75)
YA_038_gtapex_gt1.1
LTPLCVTLQCTNVTNNITDDMRGELKNCSFNMTTELRDKRQKVHALFYKLDIVPINENQNTTYRLI
NCNTASICQWPPDVSLEPEPLYLCAPKGYAILKCKDKKFNGTGPCTNVTTVQCTHGIKPVVSTQLL
LNGTLAEEEVMIRSKDIRNNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNS
TWISNTTVQGSNSTGSNDTITLPCRIKQIINMWQRNGLYVYLPPVEGVIRCVSNITGLILTRDGGS
TDSTTETFRPSGGDMRDNWRSELYKYKVVKILGGEGKWEDASVKLYPAAPPTVSPGKTGNWLTGDK
VLPTPPNPQEIHLENVTEEFNMWKNNMVDQLSEYVQEQFCLALAGSN (SEQ ID NO: 76)
YA_039_gtapex_gt1.1
LTPLCVTLQCTNVTNNITDDMRGELKNCSFNMTTELRDKRQKVHALFYKLDIVPINENQNTTYRLI
NCNTASIKQWCPDVSLEPEPLYLCAPKGYAILKCKDKKFNGTGPCTNVTTVQCTHGIKPVVSTQLL
LNGTLAEEEVMIRSKDIRNNAKNILVQFNTTVQINCTRPNNNTRKSIRIGPGQWFYATGDIIGDIR
QAHCNVTKATWNETLGKVVKQLRKHFGNNTIIRFANSTGGDLEVTTHSFNCGGEFFYCDTSGLFNS
TWISNTTVQGSNSTGSNDTITLPCRIKQIINMWQRNGLYVYLPPVEGVIRCVSNITGLILTRDGGS
TDSTTETFRPSGGDMRDNWRSELYKYKVVKILGGEGKWEDASVKLYPAAPPTVSPGKTGNWLTGDK
VLPTPPNPQEIHLENVTEEFNMWKNNMVDQLSEYVQEQFCLALAGCN (SEQ ID NO: 77)

In one embodiment of the polypeptides of any aspect disclosed herein, the non-naturally occurring HIV antigen may further a signal peptide at the amino-terminus. The signal peptide may be any signal peptide suitable for an intended purpose/expression system. In a non-limiting embodiment, the signal peptide may comprise the amino acid sequence of SEQ ID NO:50.

(SEQ ID NO: 50)
MDRAKLLLLLLLLLLPQAQA.

In another embodiment of the polypeptides of any aspect disclosed herein, fusion proteins are provided, comprising:
(a) the non-naturally occurring HIV antigen of any embodiment or combination of embodiments disclosed herein; and
(b) a protein nanoparticle component.

In this embodiment, the antigens are fused to a protein nanoparticle component, permitting display of multiple copies of the antigens on the resulting nanoparticle, permitting enhanced antigen valency that promotes stronger immune responses upon immunization with the antigen-bearing nanoparticles. Any protein nanoparticle component may be used as suitable for an intended nanoparticle system. The HIV antigen and the protein nanoparticle component may be in any orientation in the fusion protein as suitable for the particular protein nanoparticle component. In one embodiment, the protein nanoparticle component is C-terminal to the non-naturally occurring HIV antigen. In another embodiment, the protein nanoparticle component comprises a trimer-forming polypeptide. This embodiment permits optimal presentation of the antigens in view of their trimeric geometry.

In one embodiment, the protein nanoparticle component fused to the HIV antigen of the disclosure comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:51.

(SEQ ID NO: 51)
AELAYLLGELAYKLGEYRIAIRAYRIALSRDPGNAEAWYNLGNAYYKQG
RYREATYYFKKALELDPNNAEAWYNLGNAYYERGEYEEAIEYYRKALRL
DPNNADAMQNLLNAKMREE (153 dn5B).

In various embodiments, the fusion protein comprises amino acid sequence at least 50%, 55%, 60%, 65%, 70%, 75%, 80%, 85%, 90%, 92%, 94%, 96%, 98%, or 100% identical to the amino acid sequence selected from the group consisting of SEQ ID NO:52-68 and 78-95, wherein any N-terminal signal sequence of MDRAKLLLLLLLLLLPQAQA (SEQ ID NO: 50) is optional, and may be present, or may be deleted, or may be replaced with a different signal sequence. The amino acid sequences of SEQ ID NO:52-68 and 78-95 are provided in Table 3. In one embodiment, the fusion protein comprises an amino acid sequence at least 75% identical to the amino acid sequence selected from the group consisting of SEQ ID NO:52-68 and 78-95, wherein any N-terminal signal sequence of MDRAKLLLLLLLLLLPQAQA (SEQ ID NO:50) is optional, and may be present, or may be deleted, or may be replaced with a different signal sequence. In another embodiment, the fusion protein comprises an amino acid sequence at least 90% identical to the amino acid sequence selected from the group consisting of SEQ ID NO:52-68 and 78-95, wherein any N-terminal signal sequence of MDRAKLLLLLLLLLLPQAQA (SEQ ID NO:50) is optional, and may be present, or may be deleted, or may be replaced with a different signal sequence.

TABLE 3
YA_016_gtapex
LTPLCVTLQCTNVTNNITDDMRGELKNCSFNMTTELRDKRQKVHALFYKLDIVPINENQNTTYRLINCNTAQI
VQWPPDVSLEPEPIYYCAPPGYAILKCKDKKFNGTGPCTNVTTVQCTHGIKPVVSTQLLINGTLAEEEVMIRS
EDIRDNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
REGQYVYLPPVEGVIRCVSNITGLILTRDGGSTDSTTETFRPSGGDMRDNWRSELYKYKVVKKVGGEGNWEDA
SVKLFKACPPTVSPGKTGNYFTGPECPLVPPNPQEIHLENVTEEFNMWKNNMVDQVYEDVEYRFRLALGDALT
GRAPAAAEARRHHQRIEELERRQAEIEGRQAEAERELAELAYLLGELAYKLGEYRIAIRAYRIALSRDPGNAE
AWYNLGNAYYKQGRYREATYYFKKALELDPNNAEAWYNLGNAYYERGEYEEAIEYYRKALRLDPNNADAMQNL
LNAKMREE* (SEQ ID NO: 78)
MDRAKLLLLLLLLLLPQAQALTPLCVILQCTNVTNNITDDMRGELKNCSFNMTTELRDKRQKVHALFYKLDIV
PINENQNTTYRLINCNTAQIVQWPPDVSLEPEPIYYCAPPGYAILKCKDKKFNGTGPCTNVTTVQCTHGIKPV
VSTQLLINGTLAEEEVMIRSEDIRDNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNTTDDSNSTS
DDNGTITLPCRIKQIINMWQREGQYVYLPPVEGVIRCVSNITGLILTRDGGSTDSTTETFRPSGGDMRDNWRS
ELYKYKVVKKVGGEGNWEDASVKLFKACPPTVSPGKTGNYFTGPECPLVPPNPQEIHLENVTEEFNMWKNNMV
DQVYEDVEYRFRLALGDALTGRAPAAAEARRHHQRIEELERRQAEIEGRQAEAERELAELAYLLGELAYKLGE
YRIAIRAYRIALSRDPGNAEAWYNLGNAYYKQGRYREALYYFKKALELDPNNAEAWYNLGNAYYERGEYEEAI
EYYRKALRLDPNNADAMQNLLNAKMREE* (SEQ ID NO: 52)
YA_016_matureapex
TPLCVTLQCTNVSNNITDDMRGELKNCSFNMTTELRDKKQKVYSLFYRLDVVQINENQGNRTNNINKEYRLIN
CNTTQIVQWPPDVSLEPEPIYYCAPPGYAILKCKDKKFNGTGPCTNVTTVQCTHGIKPVVSTQLLINGTLAEE
EVMIRSEDIRDNAKNILVQFNTTVQINCTRPNNNTRKSIRIGPGQWFYATGDIIGDIRQAHCNVTKATWNETL
GKVVKQLRKHFGNNTIIRFANSTGGDLEVTTHSFNCGGEFFYCDTSGLFNTTDDSNSTSDDNGTITLPCRIKQ
IINMWQREGQYVYLPPVEGVIRCVSNITGLILTRDGGSTDSTTETFRPSGGDMRDNWRSELYKYKVVKKVGGE
GNWEDASVKLFKACPPTVSPGKTGNYFTGPECPLVPPNPQEIHLENVTEEFNMWKNNMVDQVYEDVEYRFRLA
LGDALTGRAPAAAEARRHHQRIEELERRQAEIEGRQAEAERELAELAYLLGELAYKLGEYRIAIRAYRIALSR
DPGNAEAWYNLGNAYYKQGRYREALYYFKKALELDPNNAEAWYNLGNAYYERGEYEEAIEYYRKALRLDPNNA
DAMQNLLNAKMREE* (SEQ ID NO: 79)
MDRAKLLLLLLLLLLPQAQALTPLCVTLQCTNVSNNITDDMRGELKNCSFNMTTELRDKKQKVYSLFYRLDVV
QINENQGNRTNNTNKEYRLINCNTTQIVQWPPDVSLEPEPIYYCAPPGYAILKCKDKKFNGTGPCTNVTTVQC
THGIKPVVSTQLLLNGTLAEEEVMIRSEDIRDNAKNILVQFNTTVQINCTRPNNNTRKSIRIGPGQWFYATGD
IIGDIRQAHCNVTKATWNETLGKVVKQLRKHFGNNTIIRFANSTGGDLEVTTHSFNCGGEFFYCDTSGLFNTT
DDSNSTSDDNGTITLPCRIKQIINMWQREGQYVYLPPVEGVIRCVSNITGLILTRDGGSTDSTTETFRPSGGD
MRDNWRSELYKYKVVKKVGGEGNWEDASVKLFKACPPTVSPGKTGNYFTGPECPLVPPNPQEIHLENVTEEFN
MWKNNMVDQVYEDVEYRFRLALGDALTGRAPAAAEARRHHQRIEELERRQAEIEGRQAEAERELAELAYLLGE
LAYKLGEYRIAIRAYRIALSRDPGNAEAWYNLGNAYYKQGRYREAIYYFKKALELDPNNAEAWYNLGNAYYER
GEYEEAIEYYRKALRLDPNNADAMQNLLNAKMREE* (SEQ ID NO: 53)
YA_020_gtapex
LTPLCVTLQCTNVTNNITDDMRGELKNCSFNMTTELRDKRQKVHALFYKLDIVPINENQNTTYRLINCNTAQI
VQWPPDVSLEPEPIYYCAPCGYAILKCKDKKFNGTGPCTNVTTVQCTHGIKPVVSTQLLLNGTLAEEEVMIRS
EDIRDNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
REGQYVYLPPVEGVIRCVSNITGLILTRDGGSTDSTTETFRPSGGDMRDNWRSELYKYKVVKKVGGEGNWEDA
SVKLFKACPPTVSPGKTGNYFTGPECPLVPPNPQEIHLENVTEEFNMWKNNMVDQVYEDVEYRFRLALGDALT
GRAPAAAEARRHHQRIEELERRQAEIECRQAEAERELAELAYLLGELAYKLGEYRIAIRAYRIALSRDPGNAE
AWYNLGNAYYKQGRYREATYYFKKALELDPNNAEAWYNLGNAYYERGEYEEAIEYYRKALRLDPNNADAMQNL
LNAKMREE* (SEQ ID NO: 80)
MDRAKLLLLLLLLLLPQAQALTPLCVTLQCTNVTNNITDDMRGELKNCSFNMTTELRDKRQKVHALFYKLDIV
PINENQNTTYRLINCNTAQIVQWPPDVSLEPEPIYYCAPCGYAILKCKDKKFNGTGPCTNVTTVQCTHGIKPV
VSTQLLLNGTLAEEEVMIRSEDIRDNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNTTDDSNSTS
DDNGTITLPCRIKQIINMWQREGQYVYLPPVEGVIRCVSNITGLILTRDGGSTDSTTETFRPSGGDMRDNWRS
ELYKYKVVKKVGGEGNWEDASVKLFKACPPTVSPGKTGNYFTGPECPLVPPNPQEIHLENVTEEFNMWKNNMV
DQVYEDVEYRFRLALGDALTGRAPAAAEARRHHQRIEELERRQAEIECRQAEAERELAELAYLLGELAYKLGE
YRIAIRAYRIALSRDPGNAEAWYNLGNAYYKQGRYREAIYYFKKALELDPNNAEAWYNLGNAYYERGEYEEAI
EYYRKALRLDPNNADAMQNLLNAKMREE* (SEQ ID NO: 54)
YA_020_matureapex
LTPLCVTLQCTNVSNNITDDMRGELKNCSFNMTTELRDKKQKVYSLFYRLDVVQINENQGNRTNNINKEYRLI
NCNTTQIVQWPPDVSLEPEPIYYCAPCGYAILKCKDKKFNGTGPCTNVTTVQCTHGIKPVVSTQLLLNGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNTTDDSNSTSDDNGTITLPCRIK
QIINMWQREGQYVYLPPVEGVIRCVSNITGLILTRDGGSTDSTTETFRPSGGDMRDNWRSELYKYKVVKKVGG
EGNWEDASVKLFKACPPTVSPGKTGNYFTGPECPLVPPNPQEIHLENVTEEFNMWKNNMVDQVYEDVEYRFRL
ALGDALTGRAPAAAEARRHHQRIEELERRQAEIECRQAEAERELAELAYLLGELAYKLGEYRIAIRAYRIALS
RDPGNAEAWYNLGNAYYKQGRYREAIYYFKKALELDPNNAEAWYNLGNAYYERGEYEEAIEYYRKALRLDPNN
ADAMQNLLNAKMREE* (SEQ ID NO: 81)
MDRAKLLLLLLLLLLPQAQALTPLCVILQCTNVSNNITDDMRGELKNCSFNMTTELRDKKQKVYSLFYRLDVV
QINENQGNRTNNTNKEYRLINCNTTQIVQWPPDVSLEPEPIYYCAPCGYAILKCKDKKFNGTGPCTNVTTVQC
THGIKPVVSTQLLINGTLAEEEVMIRSEDIRDNAKNILVQFNTTVQINCTRPNNNTRKSIRIGPGQWFYATGD
IIGDIRQAHCNVTKATWNETLGKVVKQLRKHFGNNTIIRFANSTGGDLEVTTHSFNCGGEFFYCDTSGLFNTT
DDSNSTSDDNGTITLPCRIKQIINMWQREGQYVYLPPVEGVIRCVSNITGLILTRDGGSTDSTTETFRPSGGD
MRDNWRSELYKYKVVKKVGGEGNWEDASVKLFKACPPTVSPGKIGNYFTGPECPLVPPNPQEIHLENVTEEFN
MWKNNMVDQVYEDVEYRERLALGDALTGRAPAAAEARRHHQRIEELERRQAEIECRQAEAERELAELAYLLGE
LAYKLGEYRIAIRAYRIALSRDPGNAEAWYNLGNAYYKQGRYREAIYYFKKALELDPNNAEAWYNLGNAYYER
GEYEEAIEYYRKALRLDPNNADAMQNLLNAKMREE* (SEQ ID NO: 55)
YA_023_gtapex
LTPLCVTLQCTNVTNNITDDMRGELKNCSFNMTTELRDKRQKVHALFYKLDIVPINENQNTTYRLINCNTAQI
VQWPPDVSLEPEPIYYCAPCGYAILKCKDKKFNGTGPCTNVTTVQCTHGIKPVVSTQLLINGTLAEEEVMIRS
EDIRDNAKNILVQFNTTVQINCTRPNNNTRKSIRIGPGQWFYATGDIIGDIRQARCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
REGQYVYLPPVEGVIRCVSNITGLILTRDGGSTDSTTETFRPSGGDMRDNWRSELYKYKVVKKVGGEGNWEDA
SVKLFKACPPTVSPGKIGNYFTGPECPLVPPNPQEIHLENVTEEENMWKNNMVDQVYEDVEYRFRLALGCALT
GRAPACAEARRHHQRIEELERRQAEIECRQAEAERELAELAYLLGELAYKLGEYRIAIRAYRIALSRDPGNAE
AWYNLGNAYYKQGRYREAIYYFKKALELDPNNAEAWYNLGNAYYERGEYEEAIEYYRKALRLDPNNADAMQNL
LNAKMREE* (SEQ ID NO: 82)
MDRAKLLLLLLLLLLPQAQALTPLCVTLQCTNVTNNITDDMRGELKNCSFNMTTELRDKRQKVHALFYKLDIV
PINENQNTTYRLINCNTAQIVQWPPDVSLEPEPIYYCAPCGYAILKCKDKKFNGTGPCTNVTTVQCTHGIKPV
VSTQLLLNGTLAEEEVMIRSEDIRDNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNTTDDSNSTS
DDNGTITLPCRIKQIINMWQREGQYVYLPPVEGVIRCVSNITGLILTRDGGSTDSTTETFRPSGGDMRDNWRS
ELYKYKVVKKVGGEGNWEDASVKLFKACPPTVSPGKTGNYFTGPECPLVPPNPQEIHLENVTEEFNMWKNNMV
DQVYEDVEYRFRLALGCALTGRAPACAEARRHHQRIEELERRQAEIECRQAEAERELAELAYLLGELAYKLGE
YRIAIRAYRIALSRDPGNAEAWYNLGNAYYKQGRYREAIYYFKKALELDPNNAEAWYNLGNAYYERGEYEEAI
EYYRKALRLDPNNADAMQNLLNAKMREE* (SEQ ID NO: 56)
YA_023_matureapex
LTPLCVTLQCTNVSNNITDDMRGELKNCSFNMTTELRDKKQKVYSLFYRLDVVQINENQGNRTNNTNKEYRLI
NCNTTQIVQWPPDVSLEPEPIYYCAPCGYAILKCKDKKFNGTGPCTNVTTVQCTHGIKPVVSTQLLLNGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNTTDDSNSTSDDNGTITLPCRIK
QIINMWQREGQYVYLPPVEGVIRCVSNITGLILTRDGGSTDSTTETFRPSGGDMRDNWRSELYKYKVVKKVGG
EGNWEDASVKLFKACPPTVSPGKTGNYFTGPECPLVPPNPQEIHLENVTEEFNMWKNNMVDQVYEDVEYRFRL
ALGCALTGRAPACAEARRHHQRIEELERRQAEIECRQAEAERELAELAYLLGELAYKLGEYRIAIRAYRIALS
RDPGNAEAWYNLGNAYYKQGRYREAIYYFKKALELDPNNAEAWYNLGNAYYERGEYEEAIEYYRKALRLDPNN
ADAMQNLLNAKMREE* (SEQ ID NO: 83)
MDRAKLLLLLLLLLLPQAQALTPLCVTLQCTNVSNNITDDMRGELKNCSFNMTTELRDKKQKVYSLFYRLDVV
QINENQGNRTNNTNKEYRLINCNTTQIVQWPPDVSLEPEPIYYCAPCGYAILKCKDKKFNGTGPCTNVTTVQC
THGIKPVVSTQLLLNGTLAEEEVMIRSEDIRDNAKNILVQFNTTVQINCTRPNNNTRKSIRIGPGQWFYATGD
IIGDIRQAHCNVTKATWNETLGKVVKQLRKHFGNNTIIRFANSTGGDLEVTTHSFNCGGEFFYCDTSGLFNTT
DDSNSTSDDNGTITLPCRIKQIINMWQREGQYVYLPPVEGVIRCVSNITGLILTRDGGSTDSTTETFRPSGGD
MRDNWRSELYKYKVVKKVGGEGNWEDASVKLFKACPPTVSPGKTGNYFTGPECPLVPPNPQEIHLENVTEEFN
MWKNNMVDQVYEDVEYRFRLALGCALTGRAPACAEARRHHQRIEELERRQAEIECRQAEAERELAELAYLLGE
LAYKLGEYRIAIRAYRIALSRDPGNAEAWYNLGNAYYKQGRYREAIYYFKKALELDPNNAEAWYNLGNAYYER
GEYEEAIEYYRKALRLDPNNADAMQNLLNAKMREE* (SEQ ID NO: 57)
YA_024_gtapex
LTPLCVTLQCTNVTNNITDDMRGELKNCSFNMTTELRDKRQKVHALFYKLDIVPINENQNTTYRLINCNTAQI
VQWPPDVSLEPEPIYYCAPPCYAILKCKDKKFNGTGPCTNVTTVQCTHGIKPVVSTQLLLNGTLAEEEVMIRS
EDIRDNAKNILVQFNTTVQINCTRPNNNTRKSIRIGPGQWFYATGDIIGDIRQAHCNVTKATWNETLGKVVKQ
LRKHFGNNTIIRFANSTGGDLEVITHSFNCGGEFFYCDTSGLFNTTDDSNSTSDDNGTITLPCRIKQIINMWQ
REGQYVYLPPVEGVIRCVSNITGLILTRDGGSTDSTTETFRPSGGDMRDNWRSELYKYKVVKKVGGEGNWEDA
SVKLFKACPPTVSPGKTGNYFTGPECPLVPPNPQEIHLENVTEEFNMWKNNMVDQVYEDVEYRFRLALGCALT
GRAPACAEARRHHQRIEELERRQAEIECRQAEAERELAELAYLLGELAYKLGEYRIAIRAYRIALSRDPGNAE
AWYNIGNAYYKQGRYREAIYYFKKALELDPNNAEAWYNLGNAYYERGEYEEAIEYYRKALRLDPNNADAMQNL
LNAKMREE* (SEQ ID NO: 84)
MDRAKLLLLLLLLLLPQAQALTPLCVTLQCTNVTNNITDDMRGELKNCSFNMTTELRDKRQKVHALFYKLDIV
PINENQNTTYRLINCNTAQIVQWPPDVSLEPEPIYYCAPPCYAILKCKDKKFNGTGPCTNVTTVQCTHGIKPV
VSTQLLINGTLAEEEVMIRSEDIRDNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSENCGGEFFYCDTSGLENTIDDSNSTS
DDNGTITLPCRIKQIINMWQREGQYVYLPPVEGVIRCVSNITGLILTRDGGSTDSTTETFRPSGGDMRDNWRS
ELYKYKVVKKVGGEGNWEDASVKLFKACPPTVSPGKTGNYFTGPECPLVPPNPQEIHLENVTEEFNMWKNNMV
DQVYEDVEYRERLALGCALIGRAPACAEARRHHQRIEELERRQAEIECRQAEAERELAELAYLLGELAYKLGE
YRIAIRAYRIALSRDPGNAEAWYNLGNAYYKQGRYREAIYYFKKALELDPNNAEAWYNLGNAYYERGEYEEAI
EYYRKALRLDPNNADAMQNLLNAKMREE* (SEQ ID NO: 58)
YA_024_matureapex
LTPLCVTLQCTNVSNNITDDMRGELKNCSFNMTTELRDKKQKVYSLFYRLDVVQINENQGNRINNTNKEYRLI
NCNTTQIVQWPPDVSLEPEPIYYCAPPCYAILKCKDKKFNGTGPCTNVTTVQCTHGIKPVVSTQLLINGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVITHSFNCGGEFFYCDTSGLFNITDDSNSTSDDNGTITLPCRIK
QIINMWQREGQYVYLPPVEGVIRCVSNITGLILIRDGGSTDSTTETFRPSGGDMRDNWRSELYKYKVVKKVGG
EGNWEDASVKLFKACPPTVSPGKTGNYFTGPECPLVPPNPQEIHLENVTEEENMWKNNMVDQVYEDVEYRFRE
ALGCALTGRAPACAEARRHHQRIEELERRQAEIECRQAEAERELAELAYLLGELAYKLGEYRIAIRAYRIALS
RDPGNAEAWYNLGNAYYKQGRYREAIYYFKKALELDPNNAEAWYNLGNAYYERGEYEEAIEYYRKALRLDPNN
ADAMQNLLNAKMREE* (SEQ ID NO: 85)
MDRAKLLLLLLLLLLPQAQALTPLCVTLQCTNVSNNITDDMRGELKNCSFNMTTELRDKKQKVYSLFYRLDVV
QINENQGNRTNNTNKEYRLINCNTTQIVQWPPDVSLEPEPIYYCAPPCYAILKCKDKKFNGTGPCTNVTTVQC
THGIKPVVSTQLLLNGTLAEEEVMIRSEDIRDNAKNILVQFNTTVQINCTRPNNNTRKSIRIGPGQWFYATGD
IIGDIRQAHCNVTKATWNETLGKVVKQLRKHFGNNTIIRFANSTGGDLEVTTHSFNCGGEFFYCDTSGLFNTT
DDSNSTSDDNGTITLPCRIKQIINMWQREGQYVYLPPVEGVIRCVSNITGLILTRDGGSTDSTTETFRPSGGD
MRDNWRSELYKYKVVKKVGGEGNWEDASVKLFKACPPTVSPGKTGNYFTGPECPLVPPNPQEIHLENVTEEFN
MWKNNMVDQVYEDVEYRFRLALGCALIGRAPACAEARRHHQRIEELERRQAEIECRQAEAERELAELAYLLGE
LAYKLGEYRIAIRAYRIALSRDPGNAEAWYNLGNAYYKQGRYREALYYFKKALELDPNNAEAWYNLGNAYYER
GEYEEAIEYYRKALRLDPNNADAMQNLLNAKMREE* (SEQ ID NO: 59)
YA_036_gtapex
LTPLCVTLQCTNVTNNITDDMRGELKNCSFNMTTELRDKRQKVHALFYKLDIVPINENQNTTYRLINCNTASI
KQWPPDVSLEPEPLYLCAPKGYAILKCKDKKFNGTGPCTNVTTVQCTHGIKPVVSTQLLINGTLAEEEVMIRS
EDIRDNAKNILVQFNTTVQINCTRPNNNTRKSIRIGPGQWFYATGDIIGDIRQARCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
RNGLYVYLPPVEGVIRCVSNITGLILIRDGGSTDSTTETFRPSGGDMRDNWRSELYKYKVVKILGGEGKWEDA
SVKLYPAAPPTVSPGKIGNWLTGDKVLPTPPNPQEIHLENVTEEFNMWKNNMVDQLSEYVQEQFDLALAGSNG
GSGSTAALLKALQATKARADANALEIAEIKERIRETERELAELAYLLGELAYKLGEYRIAIRAYRIALSRDPG
NAEAWYNIGNAYYKQGRYREAIYYFKKALELDPNNAEAWYNLGNAYYERGEYEEATEYYRKALRLDPNNADAM
QNLLNAKMREE* (SEQ ID NO: 60)
YA_036_matureapex
LTPLCVTLQCTNVSNNITDDMRGELKNCSFNMTTELRDKKQKVYSIFYRLDVVQINENQGNRTNNTNKEYRLI
NCNTTSIKQWPPDVSLEPEPLYLCAPKGYAILKCKDKKFNGTGPCTNVTTVQCTHGIKPVVSTQLLLNGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNTTDDSNSTSDDNGTITLPCRIK
QIINMWQRNGLYVYLPPVEGVIRCVSNITGLILTRDGGSTDSTTETFRPSGGDMRDNWRSELYKYKVVKILGG
EGKWEDASVKLYPAAPPTVSPGKTGNWLTGDKVLPTPPNPQEIHLENVTEEFNMWKNNMVDQLSEYVQEQFDL
ALAGSNGGSGSTAALLKALQATKARADANALEIAEIKERIRETERELAELAYLLGELAYKLGEYRIAIRAYRI
ALSRDPGNAEAWYNLGNAYYKQGRYREAIYYFKKALELDPNNAEAWYNLGNAYYERGEYEEAIEYYRKALRLD
PNNADAMQNLLNAKMREE* (SEQ ID NO: 61)
YA_037_gtapex
LTPLCVTLQCTNVTNNITDDMRGELKNCSFNMTTELRDKRQKVHALFYKLDIVPINENQNTTYRLINCNTASI
CQWPPDVSLEPEPLYLCAPCGYAILKCKDKKFNGTGPCTNVTTVQCTHGIKPVVSTQLLINGTLAEEEVMIRS
EDIRDNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
RNGLYVYLPPVEGVIRCVSNITGLILTRDGGSTDSTTETFRPSGGDMRDNWRSELYKYKVVKILGGEGKWEDA
SVKLYPAAPPTVSPGKTGNWLTGDKVLPTPPNPQEIHLENVTEEFNMWKNNMVDQLSEYVQEQFCLALAGSNC
GCGSCACLLKALQATKARADANALEICEIKERIRETERELAELAYLLGELAYKLGEYRIAIRAYRIALSRDPG
NAEAWYNLGNAYYKQGRYREAIYYFKKALELDPNNAEAWYNLGNAYYERGEYEEAIEYYRKALRLDPNNADAM
QNLLNAKMREE* (SEQ ID NO: 62)
YA_037_matureapex
LTPLCVTLQCTNVSNNITDDMRGELKNCSFNMTTELRDKKQKVYSLFYRLDVVQINENQGNRTNNTNKEYRLI
NCNTTSICQWPPDVSLEPEPLYLCAPCGYAILKCKDKKFNGTGPCTNVTTVQCTHGIKPVVSTQLLLNGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNTTDDSNSTSDDNGTITLPCRIK
QIINMWQRNGLYVYLPPVEGVIRCVSNITGLILTRDGGSTDSTTETFRPSGGDMRDNWRSELYKYKVVKILGG
EGKWEDASVKLYPAAPPTVSPGKTGNWLTGDKVLPTPPNPQEIHLENVTEEFNMWKNNMVDQLSEYVQEQFCL
ALAGSNCGCGSCACLLKALQATKARADANALEICEIKERIRETERELAELAYLLGELAYKLGEYRIAIRAYRI
ALSRDPGNAEAWYNLGNAYYKQGRYREAIYYFKKALELDPNNAEAWYNLGNAYYERGEYEEAIEYYRKALRLD
PNNADAMQNLLNAKMREE* (SEQ ID NO: 63)
YA_038_gtapex
LTPLCVTLQCTNVTNNITDDMRGELKNCSFNMTTELRDKRQKVHALFYKLDIVPINENQNTTYRLINCNTASI
CQWPPDVSLEPEPLYLCAPKGYAILKCKDKKFNGTGPCTNVTTVQCTHGIKPVVSTQLLLNGTLAEEEVMIRS
EDIRDNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
RNGLYVYLPPVEGVIRCVSNITGLILTRDGGSTDSTTETFRPSGGDMRDNWRSELYKYKVVKILGGEGKWEDA
SVKLYPAAPPTVSPGKTGNWLTGDKVLPTPPNPQEIHLENVTEEFNMWKNNMVDQLSEYVQEQFCLALAGSNC
GSGSTACLLKALQATKARADANALEIAEIKERIRETERELAELAYLLGELAYKLGEYRIAIRAYRIALSRDPG
NAEAWYNLGNAYYKQGRYREAIYYFKKALELDPNNAEAWYNLGNAYYERGEYEEAIEYYRKALRLDPNNADAM
QNLLNAKMREE* (SEQ ID NO: 64)
YA_038_matureapex
LTPLCVTLQCTNVSNNITDDMRGELKNCSFNMTTELRDKKQKVYSLFYRLDVVQINENQGNRTNNTNKEYRLI
NCNTTSICQWPPDVSLEPEPLYLCAPKGYAILKCKDKKFNGTGPCTNVTTVQCTHGIKPVVSTQLLLNGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNITDDSNSTSDDNGTITLPCRIK
QIINMWQRNGLYVYLPPVEGVIRCVSNITGLILIRDGGSTDSTTETFRPSGGDMRDNWRSELYKYKVVKILGG
EGKWEDASVKLYPAAPPTVSPGKTGNWLTGDKVLPTPPNPQEIHLENVTEEFNMWKNNMVDQLSEYVQEQFCL
ALAGSNCGSGSTACLLKALQATKARADANALEIAEIKERIRETERELAELAYLLGELAYKLGEYRIAIRAYRI
ALSRDPGNAEAWYNLGNAYYKQGRYREAIYYFKKALELDPNNAEAWYNLGNAYYERGEYEEAIEYYRKALRLD
PNNADAMQNLLNAKMREE* (SEQ ID NO: 65)
YA_038_gtapex_K401N
LTPLCVTLQCTNVTNNITDDMRGELKNCSFNMTTELRDKRQKVHALFYKLDIVPINENQNTTYRLINCNTASI
CQWPPDVSLEPEPLYLCAPKGYAILKCKDKKFNGTGPCTNVTTVQCTHGIKPVVSTQLLINGTLAEEEVMIRS
EDIRDNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
RNGLYVYLPPVEGVIRCVSNITGLILTRDGGSTDSTTETFRPSGGDMRDNWRSELYKYKVVKILGGEGKWEDA
SVKLYPAAPPTVSPGNTGNWLTGDKVLPTPPNPQEIHLENVTEEFNMWKNNMVDQLSEYVQEQFCLALAGSNC
GSGSTACLLKALQATKARADANALEIAEIKERIRETERELAELAYLLGELAYKLGEYRIAIRAYRIALSRDPG
NAEAWYNLGNAYYKQGRYREAIYYFKKALELDPNNAEAWYNLGNAYYERGEYEEAIEYYRKALRLDPNNADAM
QNLLNAKMREE* (SEQ ID NO: 66)
YA_039_gtapex
LTPLCVTLQCTNVTNNITDDMRGELKNCSFNMTTELRDKRQKVHALFYKLDIVPINENQNTTYRLINCNTASI
KQWCPDVSLEPEPLYLCAPKGYAILKCKDKKFNGTGPCTNVTTVQCTHGIKPVVSTQLLLNGTLAEEEVMIRS
EDIRDNAKNILVQFNTTVQINCTRPNNNTRKSIRIGPGQWFYATGDIIGDIRQARCNVTKATWNETLGKVVKQ
LRKHFGNNTIIRFANSTGGDLEVTTHSFNCGGEFFYCDTSGLFNTTDDSNSTSDDNGTITLPCRIKQIINMWQ
RNGLYVYLPPVEGVIRCVSNITGLILTRDGGSTDSTTETFRPSGGDMRDNWRSELYKYKVVKILGGEGKWEDA
SVKLYPAAPPTVSPGKTGNWLTGDKVLPTPPNPQEIHLENVTEEFNMWKNNMVDQLSEYVQEQFCLALAGCNG
GSGSTACLLKALQATKARADANALEIAEIKERIRETERELAELAYLIGELAYKLGEYRIAIRAYRIALSRDPG
NAEAWYNLGNAYYKQGRYREAIYYFKKALELDPNNAEAWYNLGNAYYERGEYEEAIEYYRKALRLDPNNADAM
QNLLNAKMREE* (SEQ ID NO: 67)
YA_039_matureapex
LTPLCVTLQCTNVSNNITDDMRGELKNCSFNMTTELRDKKQKVYSLFYRLDVVQINENQGNRTNNTNKEYRLI
NCNTTSIKQWCPDVSLEPEPLYLCAPKGYAILKCKDKKFNGTGPCTNVTTVQCTHGIKPVVSTQLLLNGTLAE
EEVMIRSEDIRDNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNTTDDSNSTSDDNGTITLPCRIK
QIINMWQRNGLYVYLPPVEGVIRCVSNITGLILTRDGGSTDSTTETFRPSGGDMRDNWRSELYKYKVVKILGG
EGKWEDASVKLYPAAPPTVSPGKTGNWLTGDKVLPTPPNPQEIHLENVTEEFNMWKNNMVDQLSEYVQEQFCL
ALAGCNGGSGSTACLLKALQATKARADANALEIAEIKERIRETERELAELAYLLGELAYKLGEYRIAIRAYRI
ALSRDPGNAEAWYNLGNAYYKQGRYREAIYYFKKALELDPNNAEAWYNLGNAYYERGEYEEAIEYYRKALRLD
PNNADAMQNLLNAKMREE (SEQ ID NO: 68)
YA_016 series:
>20M_gt11
LTPLCVTLQCTNVSNNITDDMRGELKNCSFNMTTELRDKKQKVYSLFYRLDVVQINENQGNRTNNTNKEYRLI
NCNTTQIVQWPPDVSLEPEPIYYCAPCGYAILKCKDKKFNGTGPCTNVTTVQCTHGIKPVVSTQLLLNGTLAE
EEVMIRSKDIRNNAKNILVQFNTTVQINCTRPNNNTRKSIRIGPGQWFYATGDIIGDIRQAHCNVTKATWNET
LGKVVKQLRKHFGNNTIIRFANSTGGDLEVTTHSFNCGGEFFYCDTSGLFNSTWISNTTVQGSNSTGSNDTIT
LPCRIKQIINMWQREGQYVYLPPVEGVIRCVSNITGLILTRDGGSTDSTTETFRPSGGDMRDNWRSELYKYKV
VKKVGGEGNWEDASVKLFKACPPTVSPGKTGNYFTGPECPLVPPNPQEIHLENVTEEFNMWKNNMVDQVYEDV
EYRFRLALGDALTGRAPAAAEARRHHQRIEELERRQAEIECRQAEAERELAELAYLLGELAYKLGEYRIAIRA
YRIALSRDPGNAEAWYNLGNAYYKQGRYREAIYYFKKALELDPNNAEAWYNLGNAYYERGEYEEAIEYYRKAL
RLDPNNADAMQNLLNAKMREE (SEQ ID NO: 86)
MDRAKLLLLLLLLLLPQAQALTPLCVTLQCTNVSNNITDDMRGELKNCSFNMTTELRDKKQKVYSLFYRLDVV
QINENQGNRTNNTNKEYRLINCNTTQIVQWPPDVSLEPEPIYYCAPCGYAILKCKDKKFNGTGPCTNVTTVQC
THGIKPVVSTQLLLNGTLAEEEVMIRSKDIRNNAKNILVQFNTTVQINCTRPNNNTRKSIRIGPGQWFYATGD
IIGDIRQAHCNVTKATWNETLGKVVKQLRKHFGNNTIIRFANSTGGDLEVTTHSFNCGGEFFYCDTSGLFNST
WISNTTVQGSNSTGSNDTITLPCRIKQIINMWQREGQYVYLPPVEGVIRCVSNITGLILTRDGGSTDSTTETF
RPSGGDMRDNWRSELYKYKVVKKVGGEGNWEDASVKLFKACPPTVSPGKTGNYFTGPECPLVPPNPQEIHLEN
VTEEFNMWKNNMVDQVYEDVEYRFRLALGDALTGRAPAAAEARRHHQRIEELERRQAEIECRQAEAERELAEL
AYLLGELAYKLGEYRIAIRAYRIALSRDPGNAEAWYNLGNAYYKQGRYREAIYYFKKALELDPNNAEAWYNLG
NAYYERGEYEEAIEYYRKALRLDPNNADAMQNLLNAKMREE (SEQ ID NO: 87)
>24G_gt11
LTPLCVTLQCTNVTNNITDDMRGELKNCSFNMTTELRDKRQKVHALFYKLDIVPINENQNTTYRLINCNTAQI
VQWPPDVSLEPEPIYYCAPPCYAILKCKDKKFNGTGPCTNVTTVQCTHGIKPVVSTQLLLNGTLAEEEVMIRS
KDIRNNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNSTWISNTTVQGSNSTGSNDTITLPCRIKQ
IINMWQREGQYVYLPPVEGVIRCVSNITGLILTRDGGSTDSTTETFRPSGGDMRDNWRSELYKYKVVKKVGGE
GNWEDASVKLFKACPPTVSPGKTGNYFTGPECPLVPPNPQEIHLENVTEEFNMWKNNMVDQVYEDVEYRFRLA
LGCALTGRAPACAEARRHHQRIEELERRQAEIECRQAEAERELAELAYLLGELAYKLGEYRIAIRAYRIALSR
DPGNAEAWYNLGNAYYKQGRYREAIYYFKKALELDPNNAEAWYNLGNAYYERGEYEEAIEYYRKALRLDPNNA
DAMQNLLNAKMREE* (SEQ ID NO: 88)
MDRAKLLLLLLLLLLPQAQALTPLCVTLQCTNVTNNITDDMRGELKNCSFNMTTELRDKRQKVHALFYKLDIV
PINENQNTTYRLINCNTAQIVQWPPDVSLEPEPIYYCAPPCYAILKCKDKKFNGTGPCTNVTTVQCTHGIKPV
VSTQLLLNGTLAEEEVMIRSKDIRNNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNSTWISNTTV
QGSNSTGSNDTITLPCRIKQIINMWQREGQYVYLPPVEGVIRCVSNITGLILTRDGGSTDSTTETFRPSGGDM
RDNWRSELYKYKVVKKVGGEGNWEDASVKLFKACPPTVSPGKTGNYFTGPECPLVPPNPQEIHLENVTEEFNM
WKNNMVDQVYEDVEYRFRLALGCALTGRAPACAEARRHHQRIEELERRQAEIECRQAEAERELAELAYLLGEL
AYKLGEYRIAIRAYRIALSRDPGNAEAWYNLGNAYYKQGRYREAIYYFKKALELDPNNAEAWYNLGNAYYERG
EYEEAIEYYRKALRLDPNNADAMQNLLNAKMREE* (SEQ ID NO: 89)
YA_036 series;
>36G_gt11
LTPLCVTLQCTNVTNNITDDMRGELKNCSFNMTTELRDKRQKVHALFYKLDIVPINENQNTTYRLINCNTASI
KQWPPDVSLEPEPLYLCAPKGYAILKCKDKKFNGTGPCTNVTTVQCTHGIKPVVSTQLLLNGTLAEEEVMIRS
KDIRNNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNSTWISNTTVQGSNSTGSNDTITLPCRIKQ
IINMWQRNGLYVYLPPVEGVIRCVSNITGLILTRDGGSTDSTTETFRPSGGDMRDNWRSELYKYKVVKILGGE
GKWEDASVKLYPAAPPTVSPGKTGNWLTGDKVLPTPPNPQEIHLENVTEEFNMWKNNMVDQLSEYVQEQFDLA
LAGSNGGSGSTAALLKALQATKARADANALEIAEIKERIRETERELAELAYLLGELAYKLGEYRIAIRAYRIA
LSRDPGNAEAWYNLGNAYYKQGRYREALYYFKKALELDPNNAEAWYNLGNAYYERGEYEEAIEYYRKALRLDP
NNADAMQNLLNAKMREE* (SEQ ID NO: 90)
MDRAKLLLLLLLLLLPQAQALTPLCVTLQCTNVTNNITDDMRGELKNCSFNMTTELRDKRQKVHALFYKLDIV
PINENQNTTYRLINCNTASIKQWPPDVSLEPEPLYLCAPKGYAILKCKDKKFNGTGPCTNVTTVQCTHGIKPV
VSTQLLINGTLAEEEVMIRSKDIRNNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNSTWISNTTV
QGSNSTGSNDTITLPCRIKQIINMWQRNGLYVYLPPVEGVIRCVSNITGLILTRDGGSTDSTTETFRPSGGDM
RDNWRSELYKYKVVKILGGEGKWEDASVKLYPAAPPTVSPGKTGNWLTGDKVLPTPPNPQEIHLENVTEEFNM
WKNNMVDQLSEYVQEQFDLALAGSNGGSGSTAALLKALQATKARADANALEIAEIKERIRETERELAELAYLL
GELAYKLGEYRIAIRAYRIALSRDPGNAEAWYNLGNAYYKQGRYREAIYYFKKALELDPNNAEAWYNLGNAYY
ERGEYEEAIEYYRKALRLDPNNADAMQNLLNAKMREE* (SEQ ID NO: 91)
>38G_gt11
LTPLCVTLQCTNVTNNITDDMRGELKNCSFNMTTELRDKRQKVHALFYKLDIVPINENQNTTYRLINCNTASI
CQWPPDVSLEPEPLYLCAPKGYAILKCKDKKFNGTGPCTNVTTVQCTHGIKPVVSTQLLINGTLAEEEVMIRS
KDIRNNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNSTWISNTTVQGSNSTGSNDTITLPCRIKQ
IINMWQRNGLYVYLPPVEGVIRCVSNITGLILTRDGGSTDSTTETFRPSGGDMRDNWRSELYKYKVVKILGGE
GKWEDASVKLYPAAPPTVSPGKTGNWLTGDKVLPTPPNPQEIHLENVTEEFNMWKNNMVDQLSEYVQEQFCLA
LAGSNCGSGSTACLLKALQATKARADANALEIAEIKERIRETERELAELAYLLGELAYKLGEYRIAIRAYRIA
LSRDPGNAEAWYNLGNAYYKQGRYREALYYFKKALELDPNNAEAWYNLGNAYYERGEYEEAIEYYRKALRLDP
NNADAMQNLLNAKMREE* (SEQ ID NO: 92)
MDRAKLLLLLLLLLLPQAQALTPLCVTLQCTNVTNNITDDMRGELKNCSFNMTTELRDKRQKVHALFYKLDIV
PINENQNTTYRLINCNTASICQWPPDVSLEPEPLYLCAPKGYAILKCKDKKFNGTGPCTNVTTVQCTHGIKPV
VSTQLLLNGTLAEEEVMIRSKDIRNNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNSTWISNTTV
QGSNSTGSNDTITLPCRIKQIINMWQRNGLYVYLPPVEGVIRCVSNITGLILTRDGGSIDSTTETFRPSGGDM
RDNWRSELYKYKVVKILGGEGKWEDASVKLYPAAPPTVSPGKTGNWLIGDKVLPTPPNPQEIHLENVTEEFNM
WKNNMVDQLSEYVQEQFCLALAGSNCGSGSTACLLKALQATKARADANALEIAEIKERIRETERELAELAYLL
GELAYKLGEYRIAIRAYRIALSRDPGNAEAWYNLGNAYYKQGRYREAIYYFKKALELDPNNAEAWYNLGNAYY
ERGEYEEAIEYYRKALRLDPNNADAMQNLLNAKMREE* (SEQ ID NO: 93)
>39G_gt11
LTPLCVTLQCTNVTNNITDDMRGELKNCSFNMTTELRDKRQKVHALFYKLDIVPINENQNTTYRLINCNTASI
KQWCPDVSLEPEPLYLCAPKGYAILKCKDKKFNGTGPCTNVTTVQCTHGIKPVVSTQLLLNGTLAEEEVMIRS
KDIRNNAKNILVQFNTTVQINCTRPNNNTRKSIRIGPGQWFYATGDIIGDIRQAHCNVTKATWNETLGKVVKQ
LRKHFGNNTIIRFANSTGGDLEVTTHSFNCGGEFFYCDTSGLFNSTWISNTTVQGSNSTGSNDTITLPCRIKQ
IINMWQRNGLYVYLPPVEGVIRCVSNITGLILTRDGGSTDSTTETFRPSGGDMRDNWRSELYKYKVVKILGGE
GKWEDASVKLYPAAPPTVSPGKTGNWLTGDKVLPTPPNPQEIHLENVTEEFNMWKNNMVDQLSEYVQEQFCLA
LAGCNGGSGSTACLLKALQATKARADANALEIAEIKERIRETERELAELAYLLGELAYKLGEYRIAIRAYRIA
LSRDPGNAEAWYNLGNAYYKQGRYREALYYFKKALELDPNNAEAWYNLGNAYYERGEYEEAIEYYRKALRLDP
NNADAMQNLLNAKMREE* (SEQ ID NO: 94)
MDRAKLLLLLLLLLLPQAQALTPLCVTLQCTNVINNITDDMRGELKNCSFNMTTELRDKRQKVHALFYKLDIV
PINENQNTTYRLINCNTASIKQWCPDVSLEPEPLYLCAPKGYAILKCKDKKFNGTGPCTNVTTVQCTHGIKPV
VSTQLLLNGTLAEEEVMIRSKDIRNNAKNILVQFNTTVQINCTRPNNNTRKSIRIGPGQWFYATGDIIGDIRQ
AHCNVTKATWNETLGKVVKQLRKHFGNNTIIRFANSTGGDLEVTTHSFNCGGEFFYCDTSGLFNSTWISNTTV
QGSNSTGSNDTITLPCRIKQIINMWQRNGLYVYLPPVEGVIRCVSNITGLILTRDGGSIDSTTETFRPSGGDM
RDNWRSELYKYKVVKILGGEGKWEDASVKLYPAAPPTVSPGKTGNWLTGDKVLPTPPNPQEIHLENVTEEFNM
WKNNMVDQLSEYVQEQFCLALAGCNGGSGSTACLLKALQATKARADANALEIAEIKERIRETERELAELAYLL
GELAYKLGEYRIAIRAYRIALSRDPGNAEAWYNLGNAYYKQGRYREAIYYFKKALELDPNNAEAWYNLGNAYY
ERGEYEEAIEYYRKALRLDPNNADAMQNLLNAKMREE* (SEQ ID NO: 95)

In another embodiment, the disclosure provides nanoparticles comprising multiple copies (2, 3, 4, 5, 6, 7, 8, 9, 10, 20, 60, or more) of the fusion protein of any embodiment of the disclosure. The fusion proteins may all comprise an identical HIV antigen, or may comprise different HIV antigens of the present disclosure (i.e. “mosaic” nanoparticles)

In one embodiment, the nanoparticle comprises:
(a) a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO:69-71, where residues in parentheses are optional and may be present or absent, and,
(b) a plurality of second assemblies, each second assembly comprising a plurality of fusion proteins wherein the protein nanoparticle component comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:51, or wherein the fusion protein comprises amino acid sequence at least 50%, 55%, 60%, 65%, 70%, 75%, 80%, 85%, 90%, 92%, 94%, 96%, 98%, or 100% identical to the amino acid sequence selected from the group consisting of SEQ ID NO:52-68 and 78-95, wherein any N-terminal signal sequence of MDRAKLLLLLLLLLLPQAQA (SEQ ID NO:50) is optional, and may be present, or may be deleted, or may be replaced with a different signal sequence;
wherein the plurality of first assemblies non-covalently interact with the plurality of second assemblies to form the nanoparticle; and
wherein the nanoparticle displays on its surface an immunogenic portion of the non-naturally occurring HIV antigen.

I53_dn5A*
(SEQ ID NO: 69)
(MG)KYDGSKLRIGILHARWNAEIILALVLGALKRLQEFGVKRENIIIE
TVPGSFELPYGSKLFVEKQKRLGKPLDAIIPIGVLIKGSTMHFEYICDS
TTHQLMKLNFELGIPVIFGVLTCLTDEQAEARAGLIEGKMHNHGEDWGA
AAVEMATKFN;
>I53_dn5A.1
(SEQ ID NO: 70)
(MG)KYDGSKLRIGILHARGNAEIILALVLGALKRLQEFGVKRENIIIE
TVPGSFELPYGSKLFVEKQKRIGKPLDAIIPIGVLIRGSTPHFDYIADS
TTHQLMKLNFELGIPVIFGVITADTDEQAEARAGLIEGKMHNHGEDWGA
AAVEMATKFN;
and
>I53_dn5A.2
(SEQ ID NO: 71)
(MG)KYDGSKLRIGILHARGNAEIILELVLGALKRLQEFGVKRENIIIE
TVPGSFELPYGSKLFVEKQKRIGKPLDAIIPIGVLIRGSTAHFDYIADS
TTHQLMKLNFELGIPVIFGVLTTESDEQAEERAGTKAGNHGEDWGAAAV
EMATKFN.

A plurality (2, 3, 4, 5, 6, or more) of first polypeptides self-assemble to form a first assembly, and a plurality (2, 3, 4, 5, 6, or more) of second polypeptides self-assemble to form a second assembly. A plurality of these first and second assemblies then self-assemble non-covalently via the designed interfaces to produce the nanostructures.

The number of first polypeptides in the first assemblies may be the same or different than the number of second polypeptides in the second assemblies. In one exemplary embodiment, the first assembly comprises trimers of the first polypeptides, and the second assembly comprises pentamers of the second polypeptides.

The first and second polypeptides may be of any suitable length for a given purpose of the resulting nanostructure.

The isolated polypeptides of SEQ ID NOS: 51 and 69-71 have the ability to self-assemble in pairs to form nanostructures, such as icosahedral nanostructures. The nanostructures so formed include symmetrically repeated, non-natural, non-covalent polypeptide-polypeptide interfaces that orient a first assembly and a second assembly into a nanostructure, such as one with an icosahedral symmetry. See US Published Patent Application 2022-0306697, incorporated by reference herein in its entirety.

In another aspect, the disclosure provides nucleic acids encoding the non-naturally occurring HIV antigen or fusion protein of any embodiment or combination of embodiments herein. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In one embodiment, the nucleic acid comprises mRNA. The mRNA may be modified as appropriate, for example, for use as a vaccine. In one embodiment, the RNA comprises nucleoside-modified RNA, including but not limited to NI-methylpseudouridine-5′-triphosphate containing RNA. In another embodiment, the mRNA comprises self-amplifying mRNA. In a further embodiment, the nucleic acid encodes a poly A tail (DNA) or comprises a poly A tail (RNA). In another embodiment, the nucleic acid encodes a 5′ UTR and/or a 3′ UTR (DNA) or comprises a 5′ UTR and/or a 3′ UTR (RNA).

The disclosure also provides expression vectors comprising the nucleic acid of any embodiment herein operatively linked to a suitable control element, such as a promoter. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence is still considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In one aspect, the present disclosure provides cells comprising the non-naturally occurring HIV antigen, the fusion protein, the nanoparticle, the nucleic acid, and/or the expression vector of any embodiment or combination of embodiments of the disclosure, wherein the cells can be either prokaryotic or eukaryotic, such as mammalian cells. In one embodiment, the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the disclosure is an additional part of the disclosure. The method comprises the steps of (a) culturing a host according to this aspect of the disclosure under conditions conducive to the expression of the polypeptide, and (b) optionally, recovering the expressed polypeptide.

The disclosure also provides pharmaceutical compositions and vaccines, comprising the non-naturally occurring HIV antigen, fusion protein, nanoparticle, nucleic acid, expression vector, or host cell of any preceding claim and a pharmaceutically acceptable carrier. The compositions/vaccines may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

In some embodiments, the pharmaceutical composition or vaccine comprises a nucleic acid encoding a polypeptide or fusion protein of any embodiment herein. In some such embodiments, the pharmaceutically acceptable carrier comprises a cationic lipid such as a liposome, or a cationic protein such as protamine. This embodiment is particularly useful with nucleic acid/mRNA vaccines of the disclosure.

The compositions/vaccines may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen. Exemplary adjuvants include, but are not limited to, Adju-Phos™, Adjumer™, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, BIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT (R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant B112K of Cholera Toxin mCT-B112K, p-Hydroxybenzoique acid methyl ester, pCIL-10, pCIL12, pCMVmCATI, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

In another aspect, the disclosure provides methods to treat or limit development of an HIV infection, comprising administering to a subject in need thereof an amount effective to treat or limit development of the infection of the non-naturally occurring HIV antigen, fusion protein, nanoparticle, nucleic acid, expression vector, host cell, vaccine, and/or pharmaceutical composition of any embodiment or combination of embodiments herein.

In one embodiment, the subject is not infected with HIV, wherein the administering elicits an immune response against HIV in the subject that limits development of an HIV infection in the subject.

As used herein, “limiting development” includes, but is not limited to accomplishing one or more of the following: (a) generating an immune response (antibody and/or cell-based) to HIV in the subject; (b) generating neutralizing antibodies against HIV in the subject (b) limiting build-up of HIV titer in the subject after exposure to HIV; and/or (c) limiting or preventing development of HIV symptoms after infection. Exemplary symptoms of HIV infection include, but are not limited to, fever, fatigue, swollen lymph nodes, diarrhea, weight loss, oral yeast infection, shingles, and/or pneumonia.

The administering may comprise any regimen as deemed appropriate by attending medical personnel. In one embodiment, the administering comprises administering a first dose and a second dose, wherein the second dose is administered about 2 weeks to about 12 weeks, or about 4 weeks to about 12 weeks the first dose is administered.

As used herein, an “effective amount” refers to an amount of the immunogenic composition that is effective for treating and/or limiting HIV infection. The antigens, fusion proteins, nanoparticles, nucleic acids, expression vector, cell composition, and or pharmaceutical composition, or vaccine of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the polypeptide or nanoparticle thereof. The composition can be delivered in a single bolus, or may be administered more than once (e.g., 2, 3, 4, 5, or more times) as determined by attending medical personnel.

In one embodiment, the administering comprises administering a first dose and a second dose of the immunogenic composition, wherein the second dose is administered about 2 weeks to about 12 weeks, or about 4 weeks to about 12 weeks after the first does is administered. In various further embodiments, the second dose is administered about 2, 3, 4, 5, 6, 7, 8, 9, 10, 11, or 12 weeks after the first dose. In another embodiment, three doses may be administered, with a second dose administered about 1, 2, 3, 4, 5, 6, 7, 8, 9, 10, 11, or 12 weeks after the first dose, and the third dose administered about 1, 2, 3, 4, 5, 6, 7, 8, 9, 10, 11, or 12 weeks after the second dose.

In one embodiment, the immune response comprises generation of neutralizing antibodies against HIV.

In another embodiment, the subject is infected with HIV, wherein the administering elicits an immune response against HIV in the subject that treats an HIV infection in the subject. As used herein, “treat” or “treating” includes, but is not limited to accomplishing one or more of the following: (a) reducing HIV titer in the subject; (b) limiting any increase of HIV titer in the subject; (c) reducing the severity of HIV symptoms; (d) limiting or preventing development of HIV symptoms after infection; (e) inhibiting worsening of HIV symptoms; (f) limiting or preventing recurrence of HIV symptoms in subjects that were previously symptomatic for HIV infection; and/or (e) improving survival.

The subject may be any subject that can benefit from the methods of the disclosure, including but not limited to a human subject.

### EXAMPLES

### Gp41-Free HIV-1 Env Closed Pre-Fusion Native-Like Trimeric Platforms

We describe here new trimeric platforms that exclude gp41 from HIV-1 Env immunogens while retaining closed pre-fusion native-like features.

### Design of Core Structures

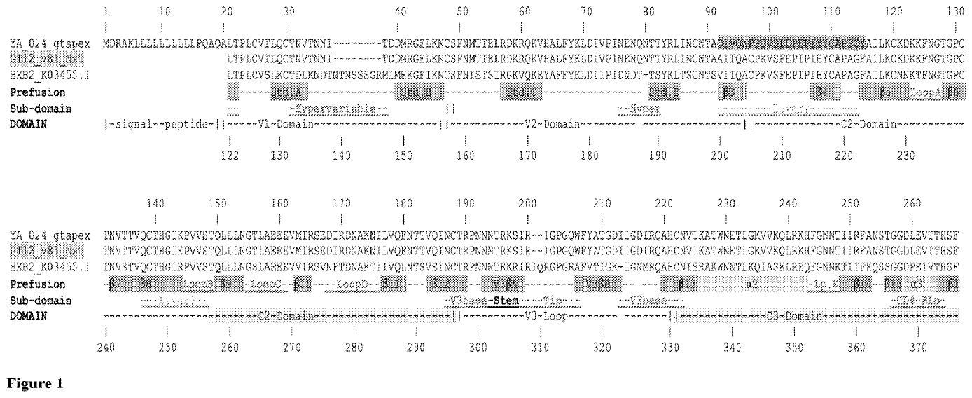

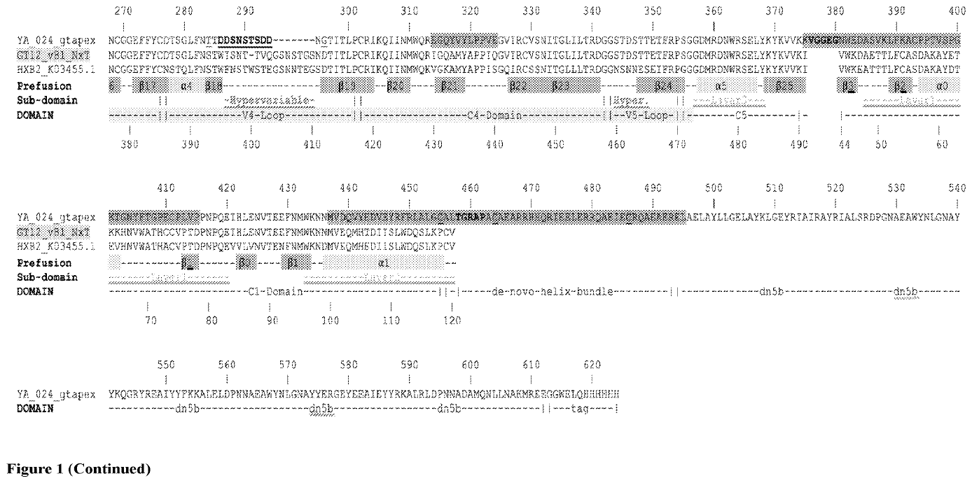

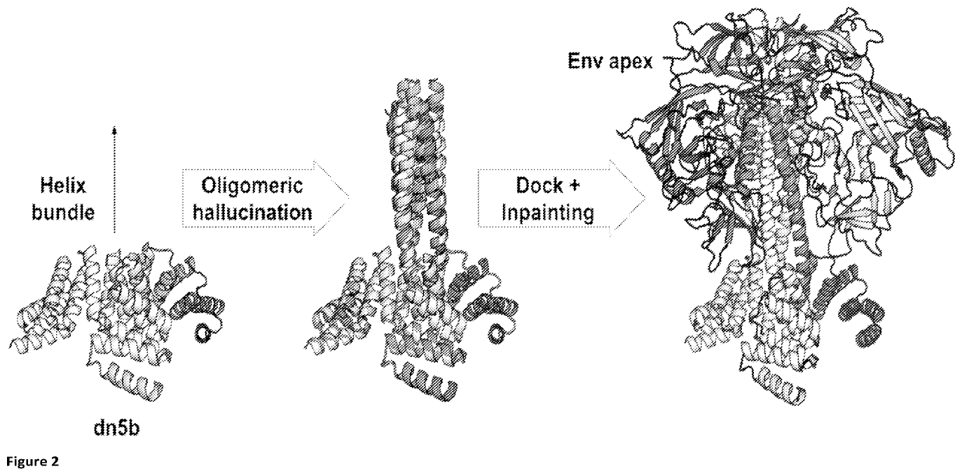

We developed a platform that preserves the trimeric geometry of Env gp120 subunits in a closed pre-fusion native-like fashion mimicking the most advanced stabilized Env trimers while removing non-neutralizing immunodominant epitopes and unstable regions of Env. Using deep learning methods developed (i.e. oligomeric hallucination, inpainting, ProteinMPNN™) together with AlphaFold2™ prediction algorithm and structure guided design, we designed new core structures to support the gp120 subunits while removing entirely the gp41 subunits which to date was not possible and would lead to not properly folded gp120 subunits. In order to present a favourable gp120 N-terminus orientation that could support the integration of a completely new trimer axis core, the gp120 of BG505 GT1.2 was permutated in a way that allows for the alpha helix 1—originally in N-term of gp120—to be placed at the gp120 C-terminus and exit at residue 120 (HXB2 numbering). The original gp120 N- and C-termini were shortened and linked with the C-terminus now preceding the original N-terminus (FIG. 1). This reloop takes place between residue 491 and 44 of gp120 (HXB2 numbering). Our platform is compatible with nanoparticle presentation which we demonstrate here with the 153_dn5 particles. Using oligomeric hallucination, helix bundles were designed with various length and backbone structures utilizing 153 dn5b particle component as an anchor for our designs (FIG. 2). These structures were then docked to the relooped trimeric BG505 GT1.2 gp120 structure. Positions were sampled over 12A and 120° to increase diversity and screened for backbone clashes. Docked structures with no backbone clashes were clustered and down-selected for inpainting to bridge the C-terminus of gp120 to the N-terminus of the de novo helix bundle (FIG. 2).

### Sequence Optimization and Stabilization

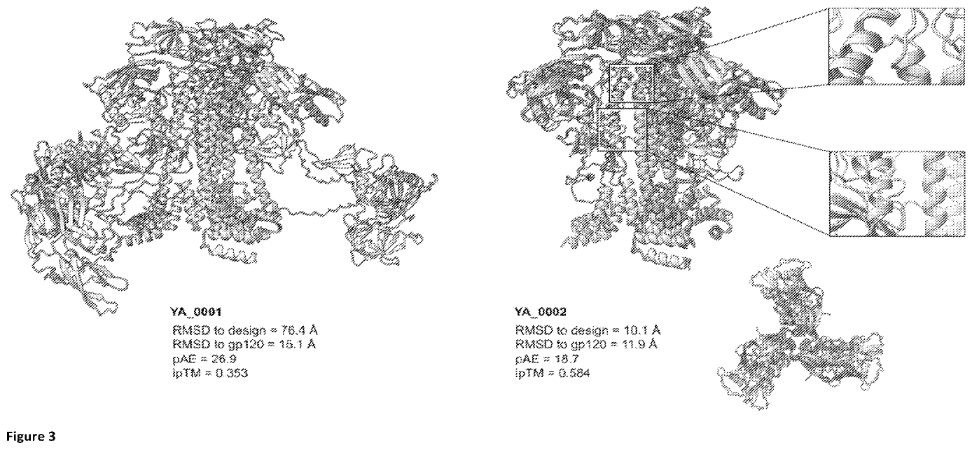

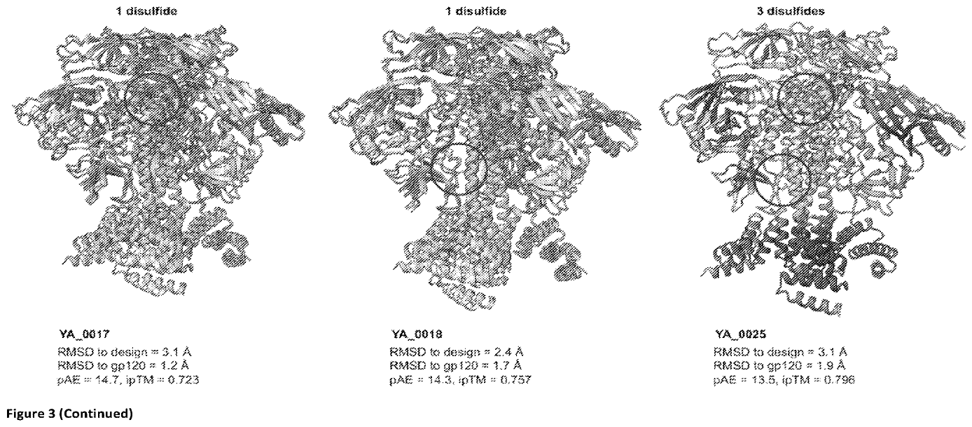

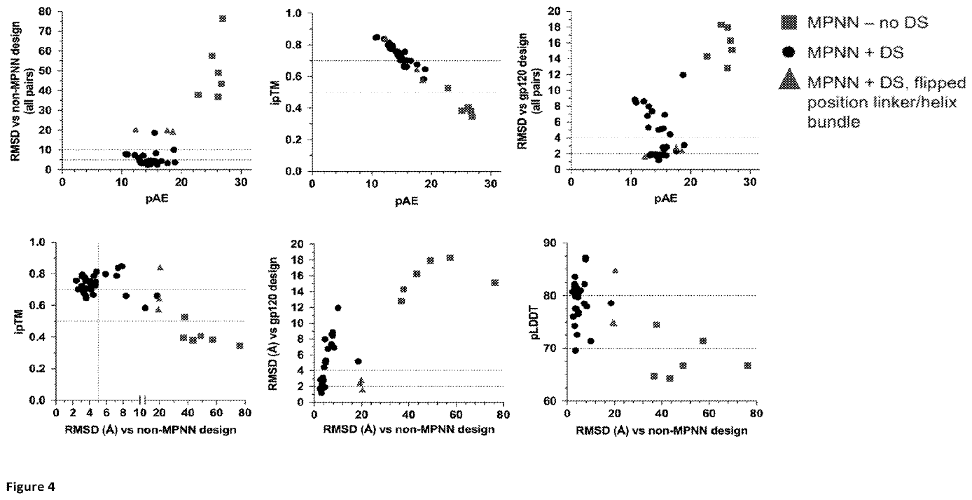

The designs were sequence optimised using ProteinMPNN™. The stretches for which the sequence was optimised using ProteinMPNN™ are highlighted in grey in FIG. 1 and include the de novo structures created as well as subdomains of Env that are part of the inner domain of the gp120. The beta 21 strand involved in the CD4 bound state bridging sheet switch was also targeted for sequence optimization with the aim of stabilising beta 21 and preventing potential reorganisation with the adjacent beta strands, including the Env beta 21 strand flip. We demonstrated that without addition of appropriate stabilisation mutations, our new Env designs could not be predicted to fold in a native conformation using AlphaFold2™ multimer (FIG. 3). Thus, in addition to predicting ProteinMPNN™ optimised designs, cysteines were introduced by structure guided design where they could potentially form disulfide bonds (DS). Single and combinations of DS were engineered followed by structure prediction using AlphaFold2™ multimer. All sequences for which DS were not included (i.e. ProteinMPNN™ parent sequences), did not have properly folded gp120s. In most cases, the de novo helix bundle together with the 153 dn5b domain folded as expected while the HIV-1 Env domains could not fold as native-like trimer apex even with 50 recycles. A number of designs containing DS were predicted to fold as native-like trimers (FIG. 3). The AlphaFold2™ scores and RMSD to the design for the best folds (FIG. 4) clearly showed the need for stabilisation mutations in order to predict type-1 fusion-de novo chimeric proteins in silico, although this might not translate to in vitro protein folding.

Designs were selected based on their folding and scoring. While designs without additional DS were not predicted to fold, we included those as controls to test whether they could fold in vitro. 26 designs were selected and ordered as series: parent design plus DS design(s). The V4 loop was re-designed in order to include 3 spaced PNGS over a 15 amino acids stretch. The V4 was designed to be 6 amino acids shorter than the original BG505 GT1.2 v8.1 V4 loop. Since these designs were heavily different compared to the original Env trimers, the apex trimerization domain may be crucial to allow for efficient protein folding in vitro. Therefore, the 26 designs, which have a germline targeting apex, were also designed with a mature trimer apex instead of the germline apex. Importantly, the N197 glycosylation site, which is knocked out in the germline targeting trimer apex, was included in mature apex designs. To aim for maximal glycan occupancy, we used the NxT optimisation approach of PNGS sequons and BG505 strain specific glycan holes 241 and 289 were filled with restored PNGS. The designs were codon optimised for Homo sapiens expression t and contain a His-tag in C-terminus for purification purposes.

From the 23 stabilisation mutations and furin-optimised cleavage site found in the most advanced SOSIP v8.1 trimers (i.e. 47D, 49E, 64K, 65K, 73C-561C, 106T, 165L, 316W, 315Q, 429R, 432Q, 500R, 501C-605C, R6 cleavage site, 519S, 535M, 543Q, 543N, I559P, 568D, 570H, 585H) only 4 mutations were maintained during the design process: 165L, 315Q, 316W, 429R. Interestingly, ProteinMPNN™ retained 64K and 47D for the 6 down-selected parent backbone designs. 65K and 432Q were retained in two separate designs.

### Expression and Identification of Gp41-Free Native-Like Trimers

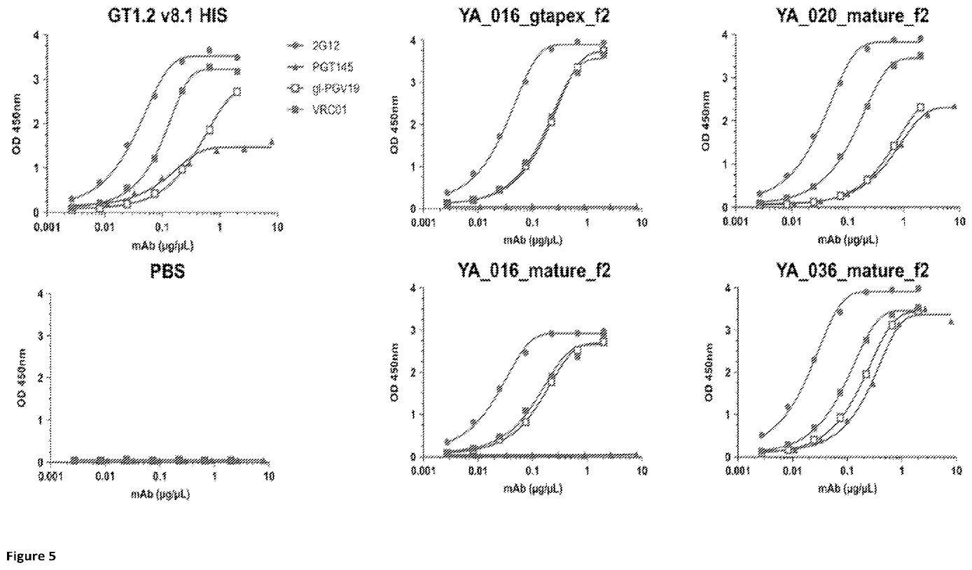

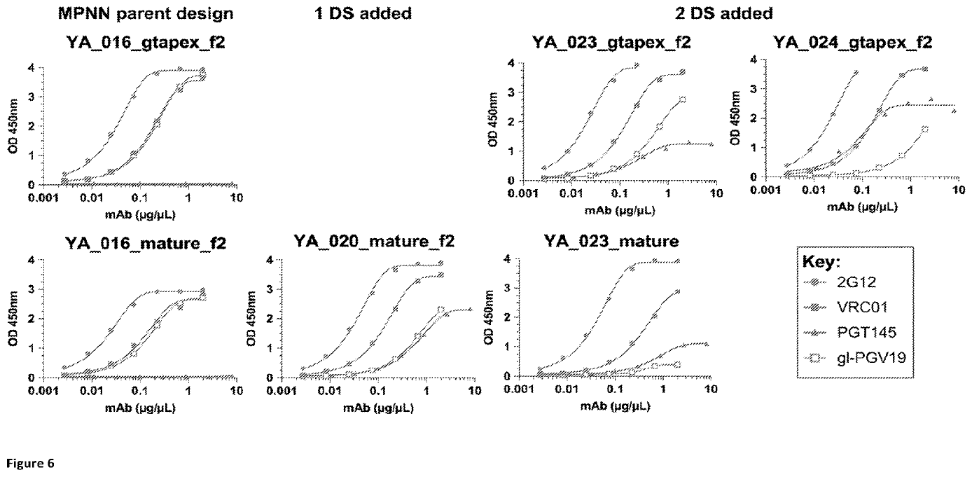

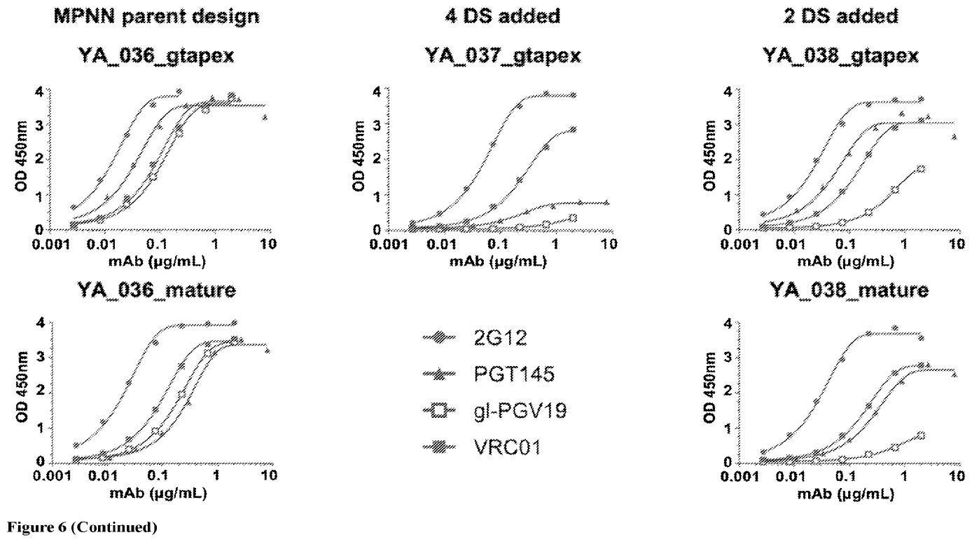

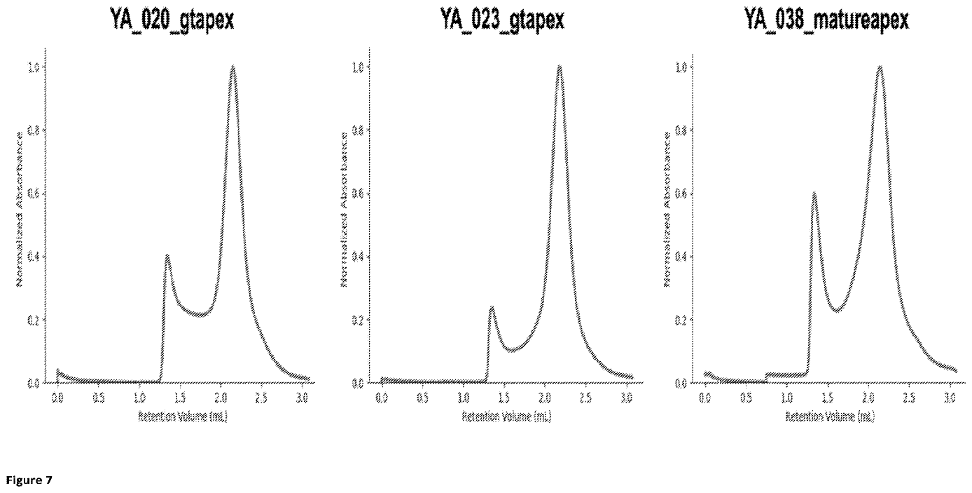

The constructs were expressed in Expi293F suspension cells and purified using nickel IMAC. All constructs expressed with yields ranging from 0.16 to 3.84 mg from 250 mL cultures. The samples were then analysed by SEC using the high throughput Superdex™ 200 Increase 5/150 GL column (Cytiva). Only putative trimer and aggregate peaks could be observed by SEC. Samples from fraction 1 and/or fraction 2 were analysed by ELISA assessing binding to a small panel of mAbs: 2G12, VRC01, PGT145 gl-PGV19. This first screen allowed to identify several designs that strongly bind PGT145, a quaternary epitope specific bNAb that only binds to closed pre-fusion native-like trimers with the appropriate high mannose trimer-associated mannose patch composition (FIG. 5). The series of constructs derived from the parent YA_001, YA_005 and YA_032 did not bind to PGT145. The YA_005 also appeared to have intrinsic misfolding and glycosylation issues as shown by low 2G12 and VRC01 binding. The YA_016 series demonstrated that disulfide bond(s) can restore binding to PGT145 while the parents YA_016_gtapex and YA_016_matureapex construct fail to fold appropriately (FIG. 6). More surprisingly, the YA_036 series showed that ProteinMPNN™ may be sufficient in some instances (FIG. 6) and additional DS modulates the antibody binding pattern compared to the parent design. Overall, we report mAb binding patterns that replicate the native-like BG505 GT1.2 v8.1 His protein and others that showed improved binding to PGT145 and gl-PGV19 compared to BG505 GT1.2 v8.1 His. Analysis by SEC using the Superose™ 6 Increase 5/150 GL column (Cytiva), which allows for better trimer resolution, showed that trimers are the predominant species following IMAC purification (FIG. 7).

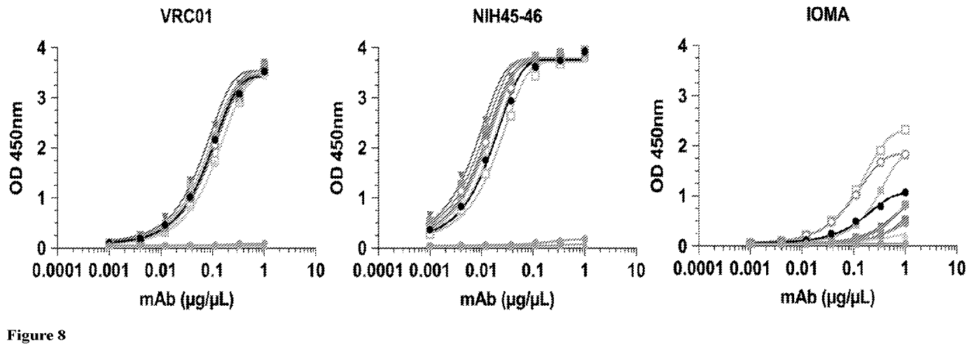

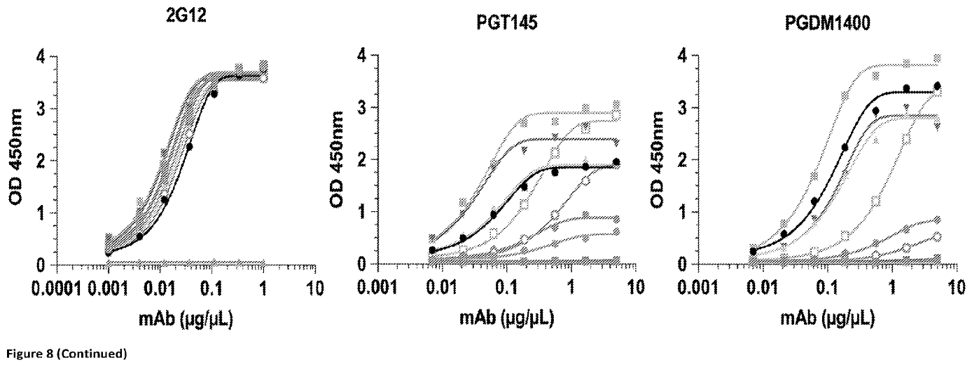

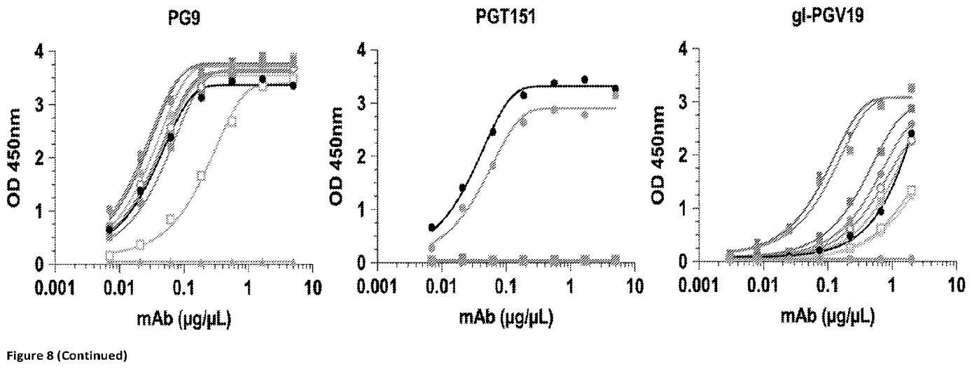

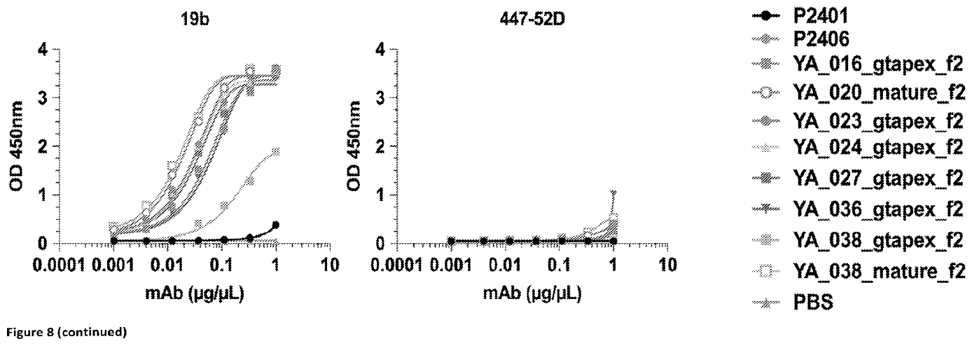

A larger panel of mAbs was then tested on the trimer fraction (f2 fraction) of designs of interests based on the ELISA screen described above ((FIG. 8). The data showed that binding to quaternary trimer apex specific PGD1400 was similar to BG505 GT1.2 v8.1 for a number of constructs (e.g. YA_036_gtapex, YA_024_gtapex), could be modulated by the germline targeting or mature apex (e.g. YA_038) or be detectable but lower than BG505 GT1.2 v8.1 (e.g. YA_020_matureapex, YA_023_gtapex). These may reflect differences in glycan composition or tightness of the trimer apex which both influence binding of PGT145 and PGDM1400. The intrinsic mannose patch appears to be preserved with high 2G12 and PGT121 binding observed for the YA_016 and YA_036 series. Binding to CD4bs bNAbs VRC01 and NIH45-46 was high for all constructs. Interestingly, we observed a range of binding to IOMA with higher binding for several designs compared to BG505 GT1.2 v8.1. The CD4bs germline targeting mAb gl-PGV19 appeared to have a range of bindings to the various designs with some reaching higher levels of binding compared to BG505 GT1.2 v8.1. Binding to the non-neutralizing mAb 447-52D, specific to the V3 loop, was absent or very low. However, 19b, a V3-specific neutralizing mAb showed high binding to all constructs indicating that the V3 is accessible in a particular way while 19b binding remains very low for GT1.2. This is an interesting point to address further since we observed high 19b binding to the membrane-anchored GT1.1 and GT1.2. Assessing V3 epitope exposure following PGT145 purification will be informative on whether a small subset of trimers exposing V3 are responsible for the observed V3 binding. In addition, we note that despite high 19b binding, strong responses were observed in knock-in mice against the CD4bs when using membrane-bound GT1.1-responses that were stronger compared to that induced by the soluble GT1.1 protein that has low 19b binding. 17b, which targets beta 20/beta 21, did not bind. However, the beta 21 strand was redesigned through ProteinMPNN™ and therefore the epitope targeted by 17b is altered. Thus, the absence of 17b binding should not be interpreted as a surrogate for well-folded trimers in this context. As expected, there was no PGT151 or 35022 binding to our designs as their binding depends on the presence of gp41. These two gp120/gp41 interface quaternary- and glycan-specific bNAbs confirm the removal of gp41 while maintaining PGT145 and PGDM1400 binding.

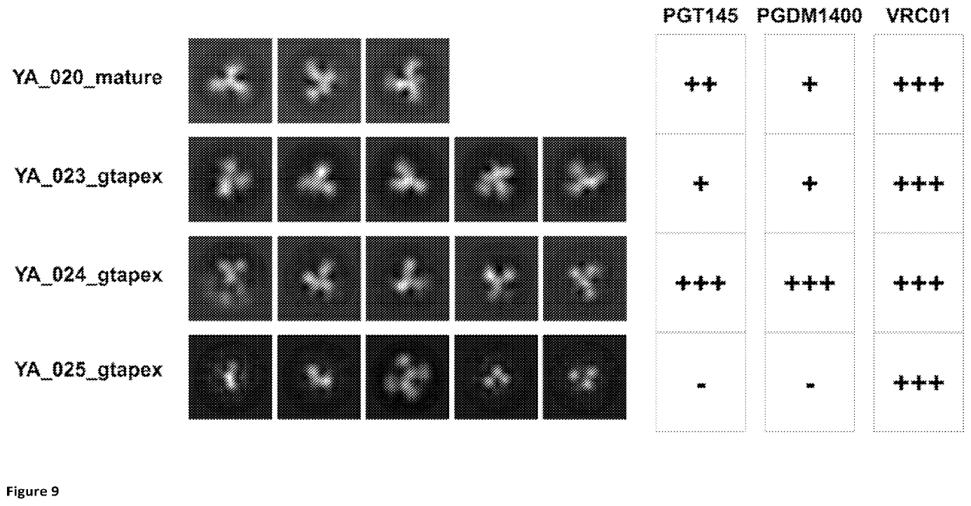

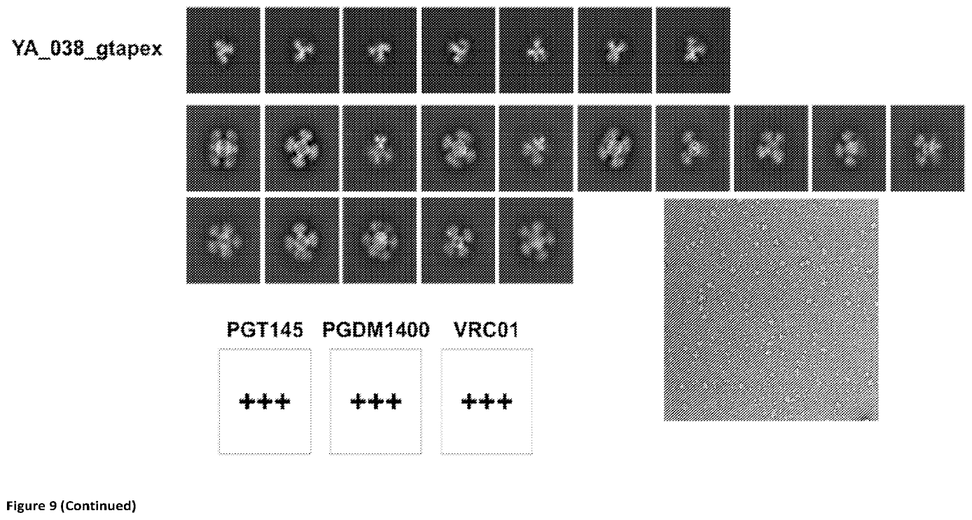

To further characterise our designs, ns-EM was carried out (FIG. 9). The 2D-class averages revealed propeller shaped native-like trimers for YA_020_matureapex, YA_023 gtapex, YA_024 gtapex and YA_038_gtapex while YA_017 gtapex, YA_018 matureapex and YA_025_gtapex were not well ordered and opened. These results are in line with the ELISA data regarding PGT145 binding. Altogether, these results indicate that gp41-free pre-fusion native-like trimers were successfully developed and with multiple core sequences. We report that YA_038_gtapex forms surprisingly well-ordered aggregates (FIG. 9) which we attribute to low affinity interaction between 153 dn5b components and His-tag mediated aggregation. Interestingly, these particles allow for the visualisation of very well ordered trimers with a rigid core and clearly identifiable gp120 domains.

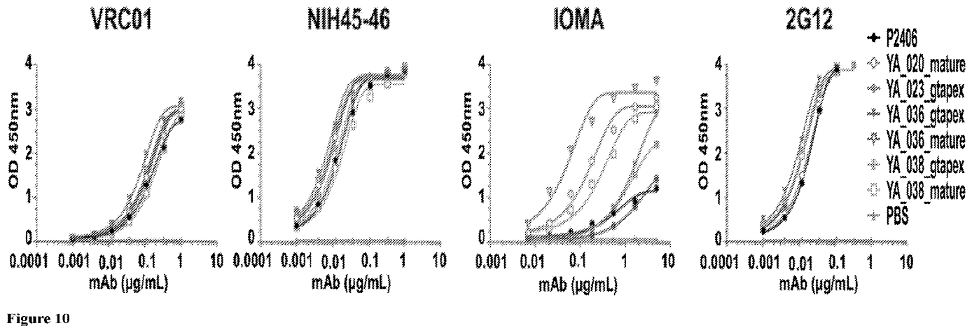

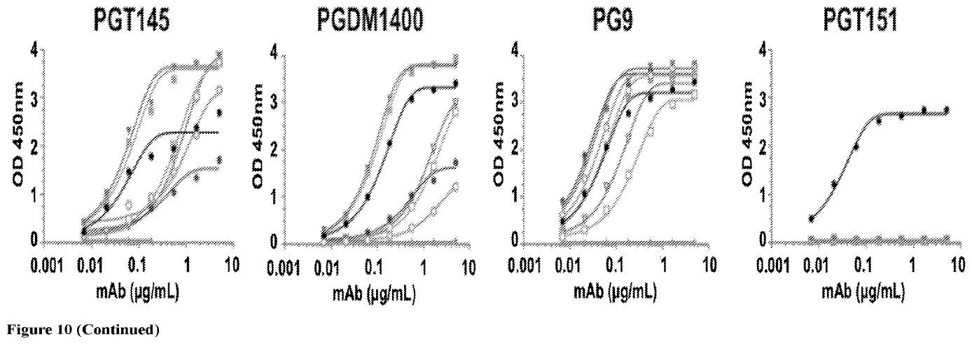

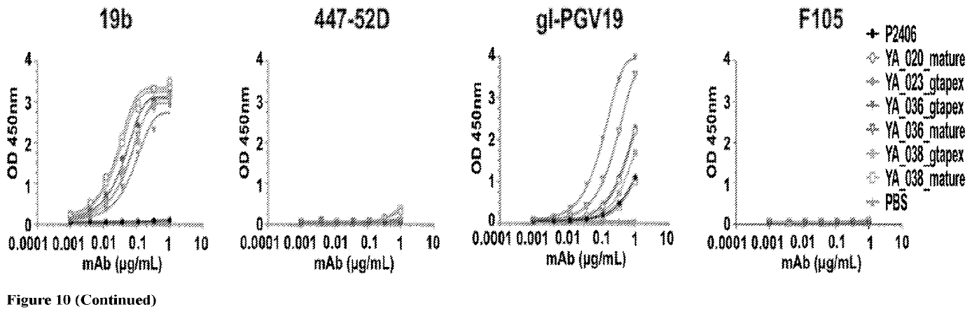

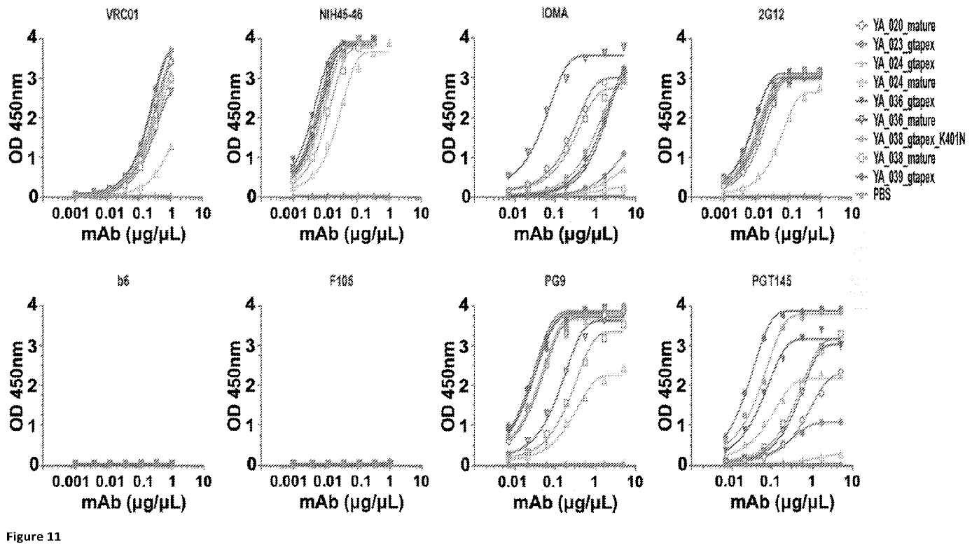

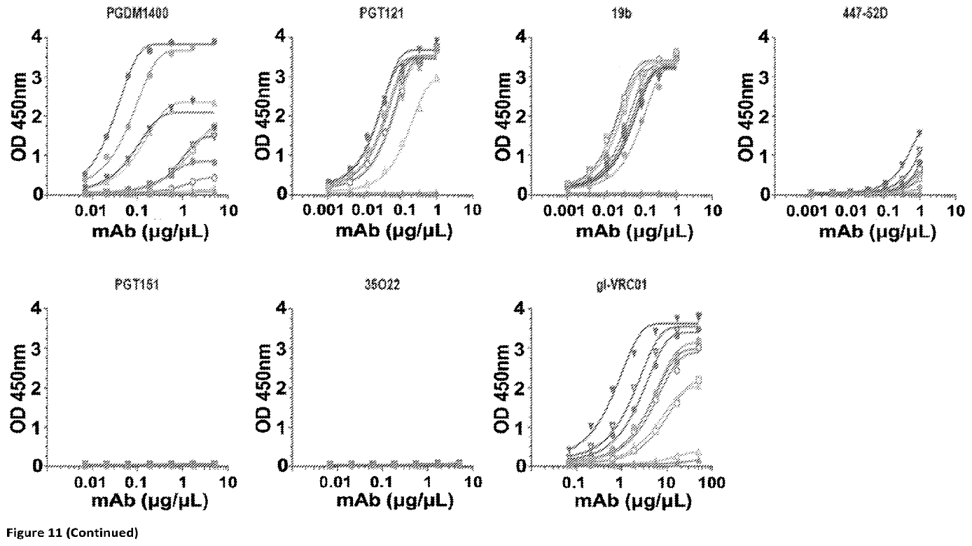

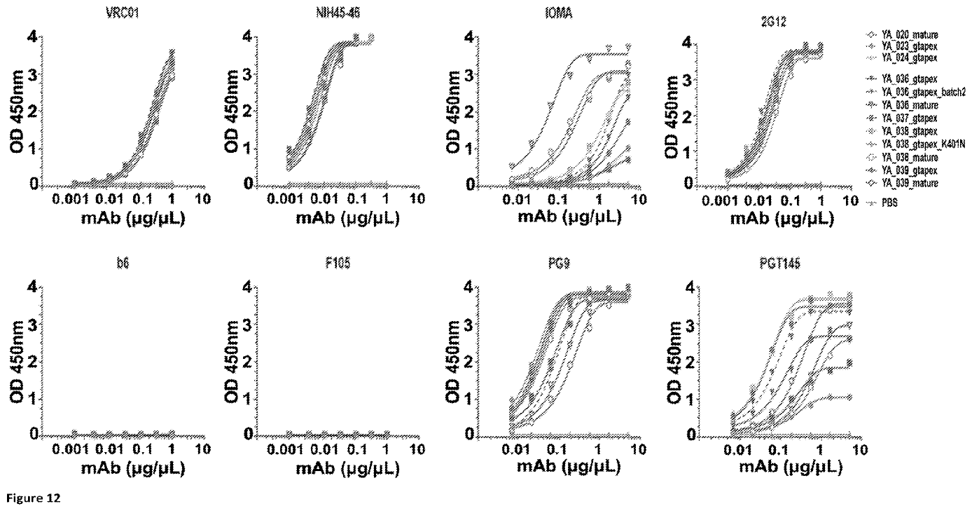

Next, we produced larger batches of proteins (800 mL cultures). Trimers were purified using IMAC and a Superose™ 6 Increase 10/300 GL. The trimers were analysed by ELISA (FIGS. 10-12). The binding profiles reported earlier were confirmed by these new data. In addition, we clearly note a marked improvement in gl-PGV19 and gl-VRC01 binding for some designs. To better assess the accessibility of the CD4 binding site and its conformation as native-like, we probed our designs against F105, a CD4bs-specific nNAb. Importantly, the epitope contact residues of F105 are preserved: K282, D368, E370, 1371, K421, G473, D474, R476. Except for a very low signal detected for YA_020_matureapex, our designs did not bind F105 suggesting that the CD4bs retains its integrity. We did not detect b6 binding, another nNAb specific to the CD4bs. 447-52D binding was detected at low levels with YA_036_gtapex and YA_036_matureapex-designs with no DS-showing the highest signal. Our results suggest our designs are efficiently mimicking and reproducing closed pre-fusion native-like trimer features while removing or preventing the presentation of non-neutralizing epitopes.

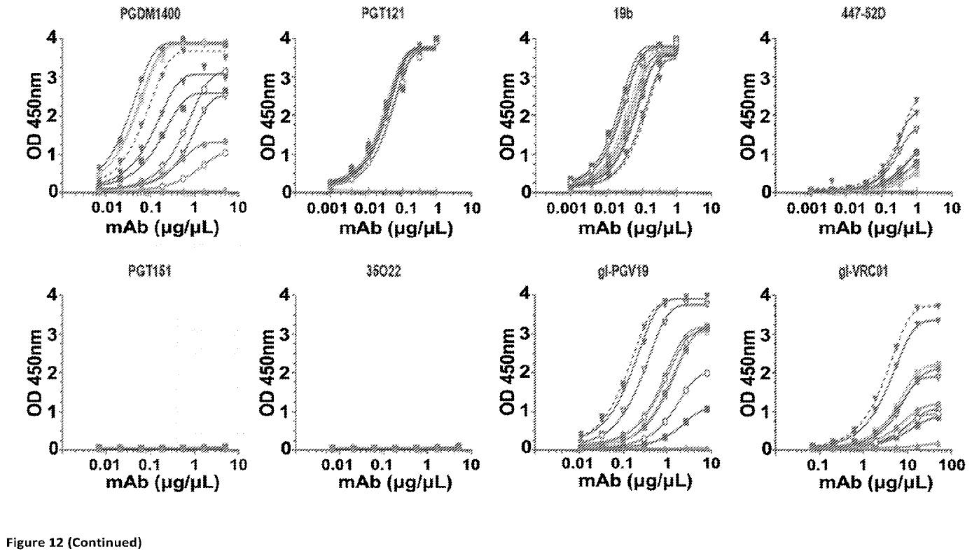

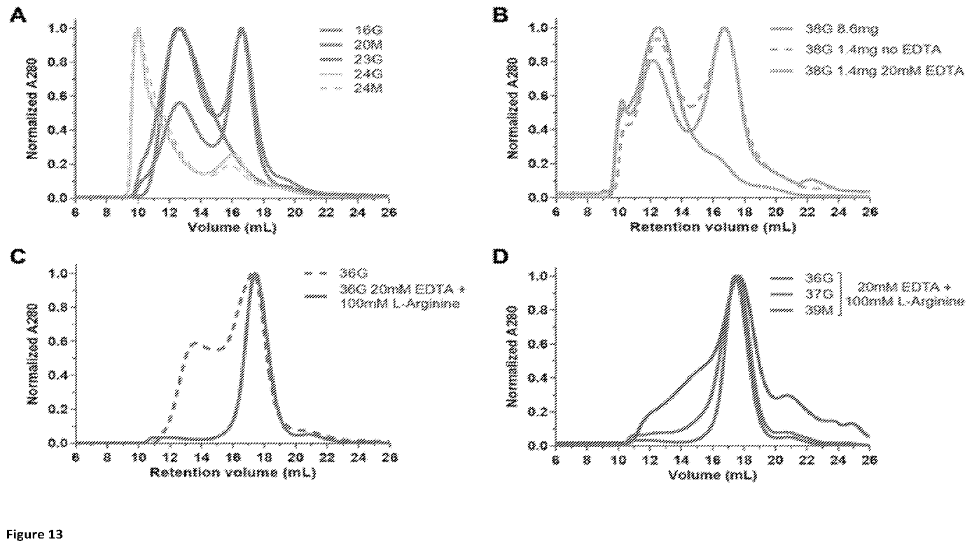

When concentrated to high concentrations, we observed aggregate formation of the designs by SEC (FIG. 13A). We showed that this aggregation is caused by weak affinity interaction mediated by His-tag and 153 dn5b that can easily be addressed by collecting IMAC eluates into a TBS solution containing EDTA and L-Arginine to a final concentration after elution of 20 mM EDTA plus 100 mM L-Arginine (FIG. 13B-D). We observed that design YA_037_gtapex presents a profile with a significant proportion of aggregates and some dimers, which could indicate improper disulfide bond formation (FIG. 13D).

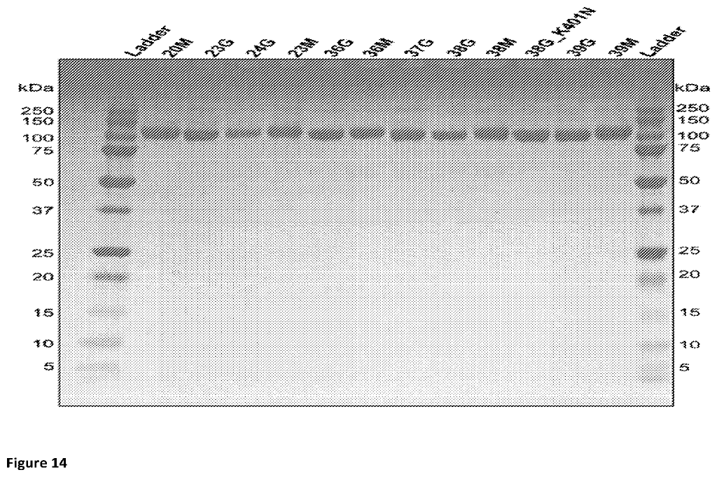

Analysis by reducing SDS-PAGE demonstrated that gp41-free Env trimer designs are very homogenous with tight migration bands (FIG. 14). We can observe slightly higher band migration patterns for mature trimers compared to their germline targeting trimer contourparts which reflects differences in apex sequences and glycosylation as there are 7 amino acids more and two additional PNGS in the mature apex compared to the germline apex.

### High Stability Profile

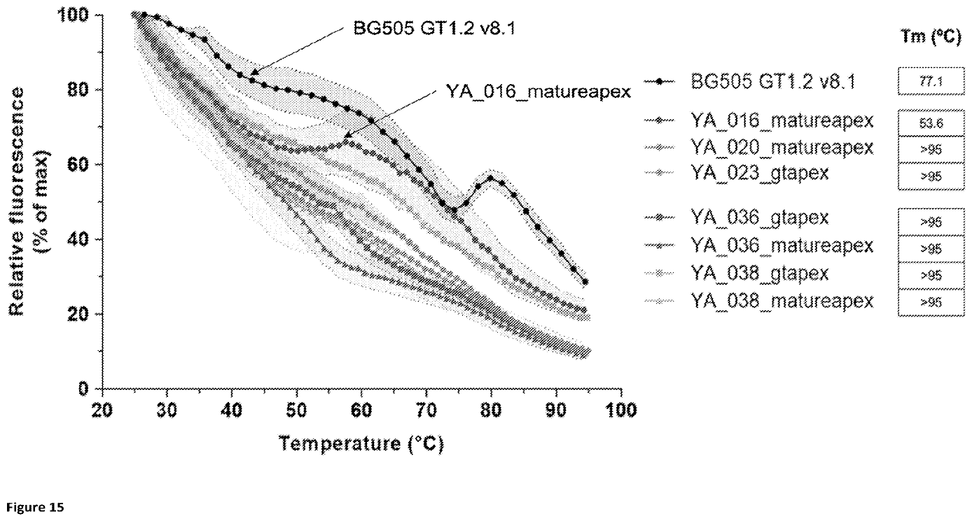

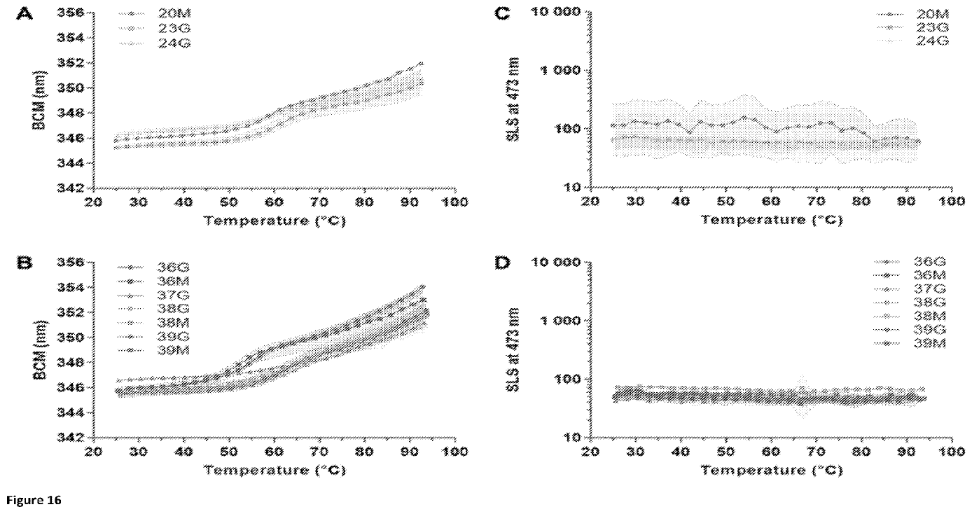

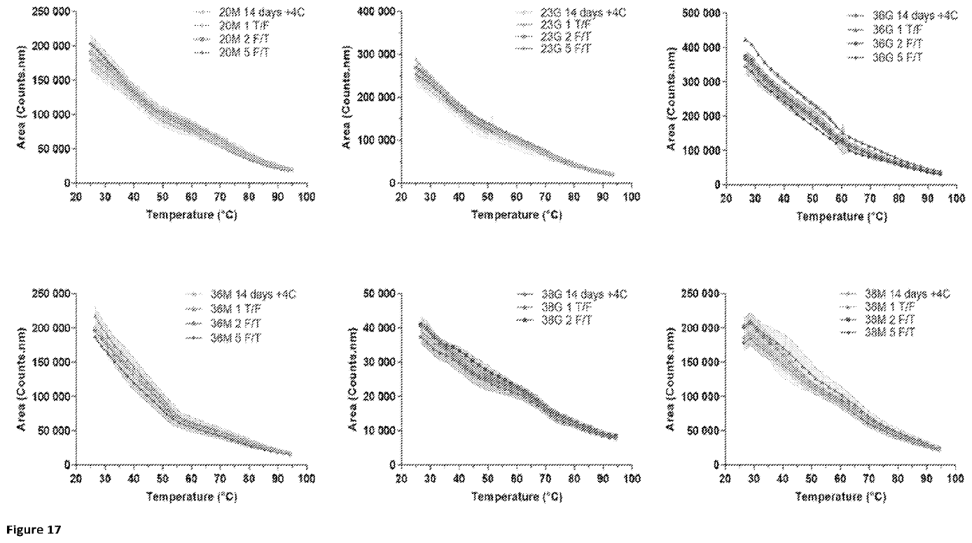

Thermostability assessment using SYPRO Orange™ (FIG. 15) also indicates that our new gp41-free Env trimer designs are extremely stable with no melting temperatures detected below 95° C. for the designs presenting native-like features and these outperform the highly stable BG505 GT1.2 v8.1 closed pre-fusion native-like trimer which melts at 77.1° C. In contrast, the YA_016 matureapex design—which does not bind to PGT145 and does not contain additional disulfide bond(s)—melted at a low temperature of 53.6° C. These results were also confirmed using tryptophane fluorescence and static light scattering with no measurable aggregation observed below 95° C. (FIG. 16). Moreover, we did not observe melting temperature below 95° C. after 1, 2 and 5 freeze/thaw cycles (samples frozen at −80° C.) for tested designs, demonstrating remarkable stability features (FIG. 17).

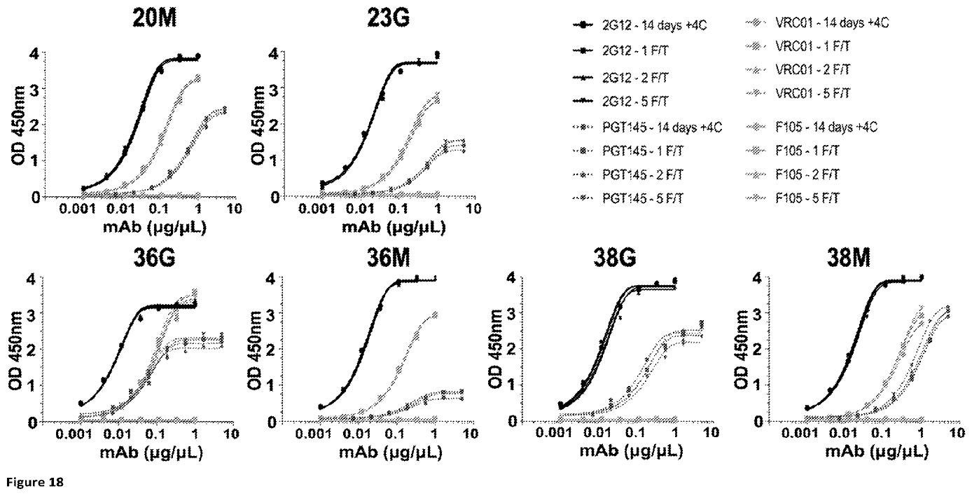

Following 1, 2 or 5 freeze/thaw cycle(s), our design did not show any sign of structural impairment with ELISA showing perfectly overlapping curves for PGT145, 2G12, VRC01 and F105 (FIG. 18). Furthermore, the melting curves remain similar with no melting temperature measurable below 95° C.

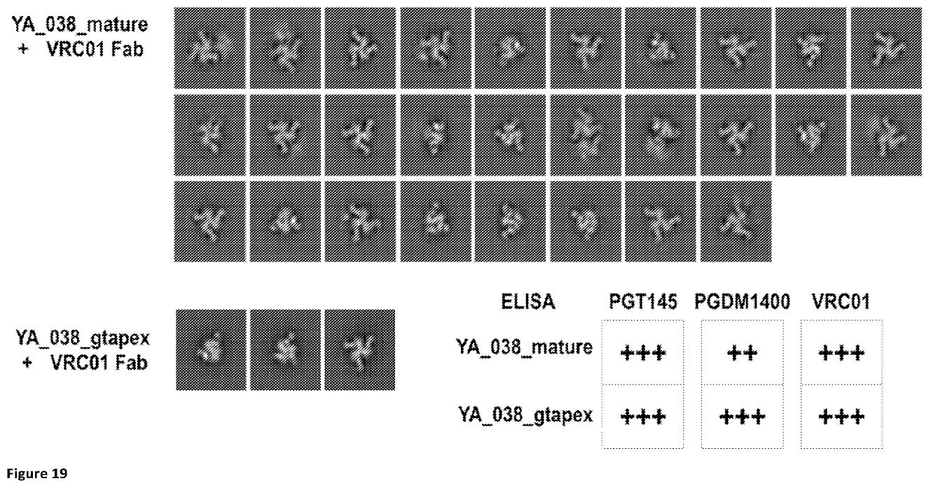

To better characterise and orient our designs in ns-EM imaging, we complexed VRC01 Fab to YA_038_maturcapex and YA_038_gtapex ((FIG. 19). The 2D-class averages showed clear closed pre-fusion native-like trimer propeller shapes with a stoichiometry of 1:3 trimer to VRC01 Fab ratio. Top, bottom and side views are identifiable.

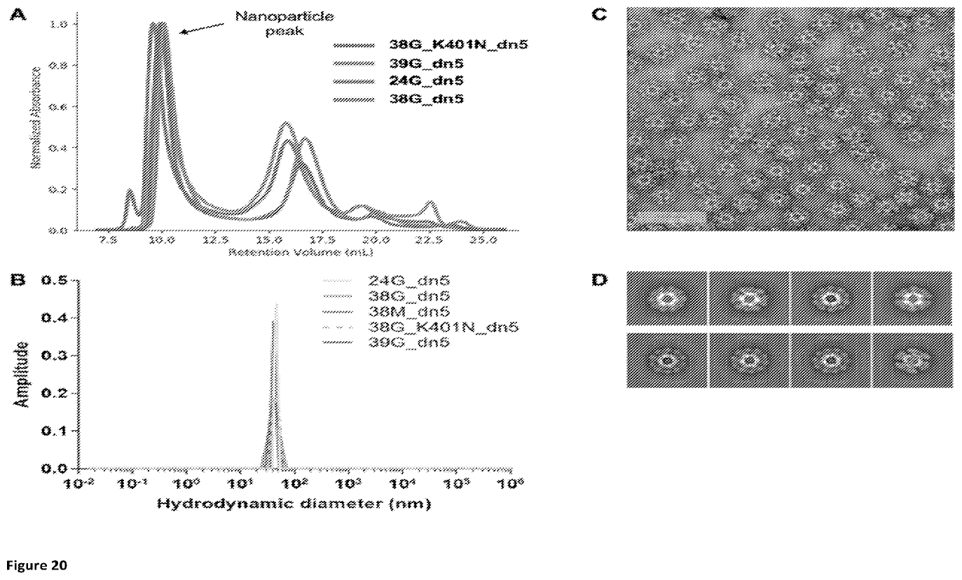

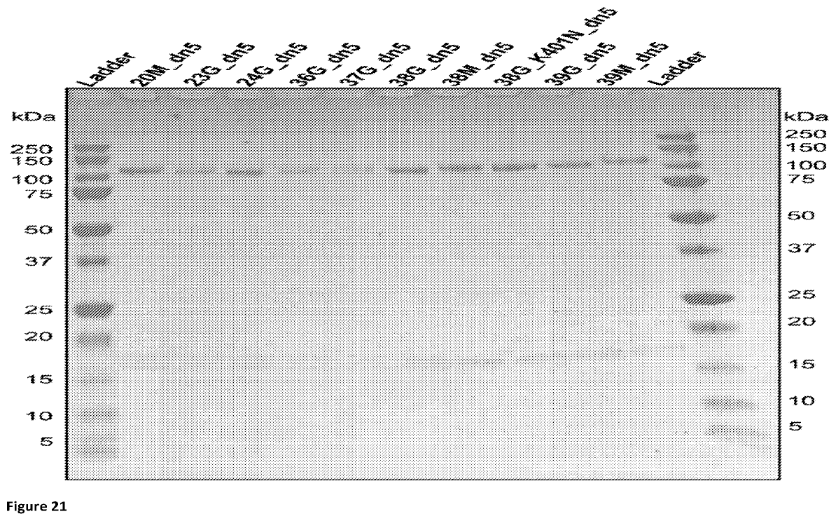

We show here that our new gp41-free trimers can be presented on nanoparticles. We assembled icosahedron nanoparticles using a 1:1 molar ration of 153-dn5A.2 pentameric component with our designs and demonstrate that they efficiently assemble (FIG. 20). NS-EM and DLS further demonstrate the well-ordered and monodispersed feature of our nanoparticles (FIG. 20B-D). We further confirmed the composition of the particles by reducing SDS-PAGE where each component of the 153_dn5 particles could be readily observed at the expected size range (FIG. 21).

We further confirmed the native-like structure of the designs using cryo-electron microscopy (cryo-EM). The cryo-EM structural analysis of YA_038_gtapex presented on 153_dn5 nanoparticle in complex with PGT145 and VRC01 Fab fragments confirmed the closed native-like pre-fusion conformation of the design. Full Fab to trimer stoichiometry was observed for VRC01 and PGT145, demonstrating the adequate presentation of those broadly neutralizing epitopes.

To further confirm adequate native-like glycosylation of the designs, glycan analysis was performed. This analysis demonstrated that immunoaffinity purified proteins (using PGT145 purification) and IMAC purified protein bad near identical glycosylation composition mimicking classic SOSIP native-like trimers, therefore improving production process compared to best in class purification methods for SOSIP native-like trimers.

### Activation of Anti-Env BCR Transgenic Ramos B-Cells

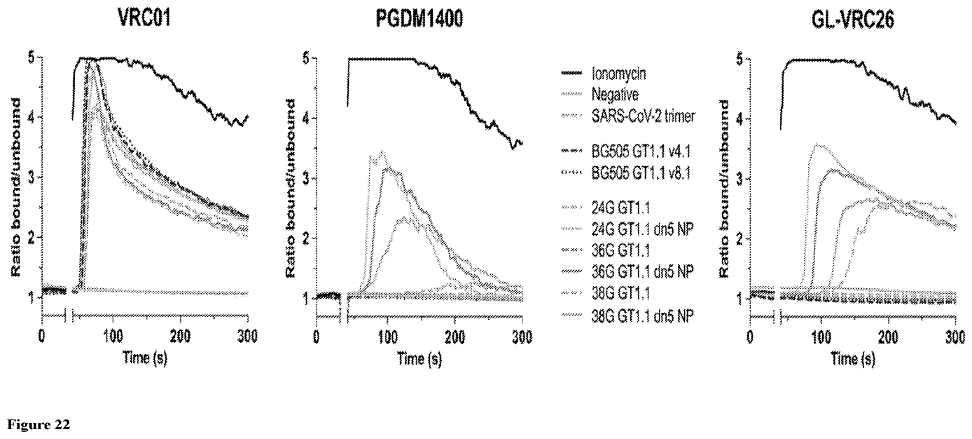

We show here that de novo designs are able to activate B cells expressing bNAb BCR, including VRC01 and PGDM1400 (FIG. 22). In addition, the new designs presented on 153_dn5 nanoparticles were able to activate germline VRC26 (GL-VRC26) precursor BCR and YA_038_gtapex gt1.1 (=38G GT1.1) which was not achieved by the SOSIP trimer control.

1. A non-naturally occurring HIV antigen, comprising the general formula X1-X2-X3-X4-X5-X6-X7-X8-X9-X10-X11, wherein:
X1 comprises an amino acid sequence at least 50%, 55%, 60%, 65%, 70%, 75%, 80%, 85%, 90%, 92%, 94%, 96%, 98%, or 100% identical to the amino acid sequence of (LTPL)CVTLQCTNVTNNITDDMRGELKNCSFNMTTELRDKRQKVHALFYKLDIVPINEN QNTTYRLINCNTA (SEQ ID NO:1), wherein residues in parentheses are optional and may be present or may be deleted;
X2 comprises an amino acid sequence at least 50%, 55%, 60%, 65%, 70%, 75%, 80%, 85%, 90%, 92%, 94%, 96%, 98%, or 100% identical to the amino acid sequence: QIVQWPPDVSLEPEPIYYCAP(P/C)(C/G)Y (SEQ ID NO:2), wherein the highlighted C residue cannot be substituted;
X3 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 3, or (SEQ ID NO:72);
X4 comprises an amino acid sequence at least 50%, 60%, 70%, 80%, 90%, 93%, or 100% identical to the amino acid sequence of SEQ ID NO:5;
X5 comprises an amino acid sequence at least 50%, 60%, 70%, 80%, 90%, 93%, or 100% identical to the amino acid sequence of SEQ ID NO:6;
X6 comprises an amino acid sequence at least 50%, 60%, 70%, 80%, 90%, 93%, or 100% identical to the amino acid sequence of SEQ ID NO:7;
X7 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 8;
X8 comprises an amino acid sequence of at least 3 contiguous amino acids identical to the amino acid sequence of SEQ ID NO:9, or at least 4 or 5, or all 6 contiguous amino acids identical to the amino acid sequence SEQ ID NO:9;
X9 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 10;
X10 comprises an amino acid sequence at least 50%, 60%, 70%, 80%, 90%, 93%, or 100% identical to the amino acid sequence of SEQ ID NO:11; and
X11 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 12.

2. The non-naturally occurring HIV antigen of claim 1, wherein:
X1 comprises the amino acid sequence: (LTPL)CVTLQCTNVTNNITDDMRGELKNCSFNMTTELRDKRQKVHALFYKLDIVPINEN QNTTYRLINCNTA (SEQ ID NO:1), wherein residues in parentheses are optional and may be present or may be deleted;
X2 comprises the amino acid sequence: QIVQWPPDVSLEPEPIYYCAP(P/C)(C/G)Y (SEQ ID NO:2), wherein the highlighted C residue cannot be substituted;
X4 comprises the amino acid sequence of SEQ ID NO:4 or SEQ ID NO:5;
X6 comprises the amino acid sequence of SEQ ID NO:7;
X8 comprises the amino acid sequence of SEQ ID NO:9;
X9 comprises the amino acid sequence of SEQ ID NO: 10; and
X11 comprises the amino acid sequence of SEQ ID NO:12.

3. The non-naturally occurring HIV antigen of claim 1, wherein:
X1 comprises the amino acid sequence: (LTPL)CVTLQCTNVTNNITDDMRGELKNCSFNMTTELRDKRQKVHALFYKLDIVPINEN QNTTYRLINCNTA (SEQ ID NO:1), wherein residues in parentheses are optional and may be present or may be deleted;
X2 comprises the amino acid sequence: QIVQWPPDVSLEPEPIYYCAP(P/C)(C/G)Y (SEQ ID NO:2), wherein the highlighted C residue cannot be substituted;
X3 comprises the amino acid sequence of SEQ ID NO:3, or SEQ ID NO:72;
X4 comprises the amino acid sequence of SEQ ID NO:4 or SEQ ID NO:5;
X5 comprises the amino acid sequence of SEQ ID NO:6;
X6 comprises the amino acid sequence of SEQ ID NO:7;
X7 comprises the amino acid sequence of SEQ ID NO:8;
X8 comprises the amino acid sequence of SEQ ID NO:9;
X9 comprises the amino acid sequence of SEQ ID NO:10;
X10 comprises the amino acid sequence of SEQ ID NO:11; and
X11 comprises the amino acid sequence of SEQ ID NO: 12, or comprises the amino acid sequence of SEQ ID NO: 13 or 14.

4-5. (canceled)

6. The non-naturally occurring HIV antigen of claim 1; comprising the general formula X1-X2-X3-X4-X5-X6-X7-X8-X9-X10-X11-X12-X13, wherein X1-X11 are as defined in claims 1-5, and wherein
X12 comprises an amino acid sequence at least 20%, 40%, 60%, 89%, or 100% identical to the amino acid sequence of SEQ ID NO:15; and
X13 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 16.

7. The non-naturally occurring HIV antigen of claim 6, wherein
X12 comprises the amino acid sequence of SEQ ID NO: 15; and
X13 comprises the amino acid sequence of SEQ ID NO: 16, or comprises the amino acid sequence selected from the group consisting of SEQ ID NO: 17-19.

8-9. (canceled)

10. The non-naturally occurring HIV antigen of claim 1, comprising an amino acid sequence at least 50%, 55%, 60%, 65%, 70%, 75%, 80%, 85%, 90%, 92%, 94%, 96%, 98%, or 100% identical to the amino acid sequence the amino acid sequence selected from the group consisting of SEQ ID NO:20-27 and 73-74.

11. A non-naturally occurring HIV antigen, comprising the general formula X1-X2-X3-X4-X5-X6-X7-X8-X9-X10-X11, wherein:
X1 comprises an amino acid sequence at least 50%, 55%, 60%, 65%, 70%, 75%, 80%, 85%, 90%, 92%, 94%, 96%, 98%, or 100% identical to the amino acid sequence of (LTPL)CVTLQCTNVTNNITDDMRGELKNCSFNMTTELRDKRQKVHALFYKLDIVPINEN QNTTYRLINCNTA (SEQ ID NO:1), or (LTPL)CVTLQCTNVSNNITDDMRGELKNCSFNMTTELRDKKQKVYSLFYRLDVVQINEN QGNRTNNTNKEYRLINCNTT (SEQ ID NO:28), wherein residues in parentheses are optional and may be present or may be deleted;
X2 comprises an amino acid sequence at least 50%, 55%, 60%, 65%, 70%, 75%, 80%, 85%, 90%, 92%, 94%, 96%, 98%, or 100% identical to the amino acid sequence: SI(K/C)QW(P/C)PDVSLEPEPLYLCAP(K/C)GY (SEQ ID NO:29), wherein the highlighted C residue cannot be substituted;
X3 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 3 or SEQ ID NO:72;
X4 comprises an amino acid sequence at least 50%, 60%, 70%, 80%, 90%, 93%, or 100% identical to the amino acid sequence of SEQ ID NO:4 or 5;
X5 comprises an amino acid sequence at least 50%, 60%, 70%, 80%, 90%, 93%, or 100% identical to the amino acid sequence of SEQ ID NO:6;
X6 comprises an amino acid sequence at least 50%, 60%, 70%, 80%, 90%, 93%, or 100% identical to the amino acid sequence of SEQ ID NO:30;
X7 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 8;
X8 comprises an amino acid sequence of at least 3 contiguous amino acids identical to the amino acid sequence of SEQ ID NO:31, or at least 4 or all 5 contiguous amino acids identical to the amino acid sequence of SEQ ID NO:31;
X9 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 32;
X10 comprises an amino acid sequence at least 50%, 60%, 70%, 80%, 90%, 93%, or 100% identical to the amino acid sequence of SEQ ID NO:11; and
X11 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO: 33.

12. The non-naturally occurring HIV antigen of claim 11, wherein:
X1 comprises the amino acid sequence: (LTPL)CVTLQCTNVTNNITDDMRGELKNCSFNMTTELRDKRQKVHALFYKLDIVPINEN QNTTYRLINCNTA (SEQ ID NO:1); or (LTPL)CVTLQCTNVSNNITDDMRGELKNCSFNMTTELRDKKQKVYSLFYRLDVVQINEN QGNRTNNTNKEYRLINCNTT (SEQ ID NO:28), wherein residues in parentheses are optional and may be present or may be deleted
X2 comprises the amino acid sequence: SI(K/C)QW(P/C)PDVSLEPEPLYLCAP(K/C)GY (SEQ ID NO:29), wherein the highlighted C residue cannot be substituted;
X4 comprises the amino acid sequence of SEQ ID NO:4 or 5;
X6 comprises the amino acid sequence of SEQ ID NO:30;
X8 comprises the amino acid sequence of SEQ ID NO:31;
X9 comprises the amino acid sequence of SEQ ID NO:34); and
X11 comprises the amino acid sequence of SEQ ID NO:33.

13. The non-naturally occurring HIV antigen of claim 11, wherein:
X1 comprises the amino acid sequence: (LTPL)CVTLQCTNVTNNITDDMRGELKNCSFNMTTELRDKRQKVHALFYKLDIVPINEN QNTTYRLINCNTA (SEQ ID NO:1), wherein residues in parentheses are optional and may be present or may be deleted
X2 comprises the amino acid sequence: SI(K/C)QW(P/C)PDVSLEPEPLYLCAP(K/C)GY (SEQ ID NO:29), wherein the highlighted C residue cannot be substituted;
X3 comprises the amino acid sequence of SEQ ID NO:3 or SEQ ID NO:72;
X4 comprises the amino acid sequence of SEQ ID NO:4 or 5;
X5 comprises the amino acid sequence of SEQ ID NO:6;
X6 comprises the amino acid sequence of SEQ ID NO:30;
X7 comprises the amino acid sequence of SEQ ID NO:8;
X8 comprises the amino acid sequence of SEQ ID NO:31;
X9 comprises the amino acid sequence of SEQ ID NO:34;
X10 comprises the amino acid sequence of SEQ ID NO:11; and
X11 comprises the amino acid sequence of SEQ ID NO:33.

14. The non-naturally occurring HIV antigen of claim 11, wherein X11 comprises the amino acid sequence of SEQ ID NO:35 or 36.

15. (canceled)

16. The non-naturally occurring HIV antigen of claim 11; comprising the general formula X1-X2-X3-X4-X5-X6-X7-X8-X9-X10-X11-X12-X13, wherein X1-X11 are as defined in claims 11-15, and wherein
X12 comprises an amino acid sequence at least 50% or 100% identical to the amino acid sequence: (G/C)G(S/C)G; and
X13 comprises an amino acid sequence at least 50%, 55%, 60%, 65%, 70%, 75%, 80%, 85%, 90%, 92%, 94%, 96%, 98%, or 100% identical to the amino acid sequence of SEQ ID NO:37.

17. The non-naturally occurring HIV antigen of claim 16, wherein
X12 comprises the amino acid sequence: (G/C)G(S/C)G, or comprises the amino acid sequence GGSG (SEQ ID NO: 96), or CGCG (SEQ ID NO: 97), or CGSG (SEQ ID NO. 98; and
X13 comprises the amino acid sequence of SEQ ID NO:37, or comprises the amino acid sequence selected from the group consisting of SEQ ID NO:38-40.

18-19. (canceled)

20. The non-naturally occurring HIV antigen of claim 11, comprising an amino acid sequence at least 50%, 55%, 60%, 65%, 70%, 75%, 80%, 85%, 90%, 92%, 94%, 96%, 98%, or 100% identical to the amino acid sequence selected from the group consisting of SEQ ID NO:41-49 and 75-77.

21-22. (canceled)

23. A fusion protein, comprising:
(a) the non-naturally occurring HIV antigen of claim 1; and
(b) a protein nanoparticle component.

24-27. (canceled)

28. A nanoparticle comprising multiple copies of the fusion protein of claim 23.

29. (canceled)

30. A nucleic acid encoding the non-naturally occurring HIV antigen of claim 1.

31-36. (canceled)

37. An expression vector comprising the nucleic acid of claim 30 operatively linked to a suitable control element, such as a promoter.

38. A host cell comprising the expression vector of claim 37.

39. A pharmaceutical composition or vaccine, comprising the non-naturally occurring HIV antigen of claim 1, and a pharmaceutically acceptable carrier.

40-41. (canceled)

42. A method to treat or limit development of an HIV infection, comprising administering to a subject in need thereof an amount effective to treat or limit development of the infection of the non-naturally occurring HIV antigen of claim 1.

43-46. (canceled)


SEQUENCE LISTING (102 sequences)

SEQ ID NO: 1 - protein, 71 aa, synthetic construct
  VARIANT 1..4: note = optionally absent
LTPLCVTLQCTNVTNNITDDMRGELKNCSFNMTTELRDKRQKVHALFYKLDIVPINENQNTTYRLINCNTA

SEQ ID NO: 2 - protein, 24 aa, synthetic construct
  VARIANT 22: note = X is P or C
  VARIANT 23: note = X is C or G
QIVQWPPDVSLEPEPIYYCAPXXY

SEQ ID NO: 3 - protein, 167 aa, synthetic construct
AILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLF

SEQ ID NO: 4 - protein, 15 aa, synthetic construct
NTTDDSNSTSDDNGT

SEQ ID NO: 5 - protein, 21 aa, synthetic construct
NSTWISNTTVQGSNSTGSNDT

SEQ ID NO: 6 - protein, 16 aa, synthetic construct
ITLPCRIKQIINMWQR

SEQ ID NO: 7 - protein, 11 aa, synthetic construct
EGQYVYLPPVE

SEQ ID NO: 8 - protein, 50 aa, synthetic construct
GVIRCVSNITGLILTRDGGSTDSTTETFRPSGGDMRDNWRSELYKYKVVK

SEQ ID NO: 9 - protein, 6 aa, synthetic construct
KVGGEG

SEQ ID NO: 10 - protein, 35 aa, synthetic construct
NWEDASVKLFKACPPTVSPGKTGNYFTGPECPLVP

SEQ ID NO: 11 - protein, 21 aa, synthetic construct
PNPQEIHLENVTEEFNMWKNN

SEQ ID NO: 12 - protein, 21 aa, synthetic construct
  VARIANT 19: note = X is D or C
MVDQVYEDVEYRFRLALGXAL

SEQ ID NO: 13 - protein, 21 aa, synthetic construct
MVDQVYEDVEYRFRLALGCAL

SEQ ID NO: 14 - protein, 21 aa, synthetic construct
MVDQVYEDVEYRFRLALGDAL

SEQ ID NO: 15 - protein, 5 aa, synthetic construct
TGRAP

SEQ ID NO: 16 - protein, 33 aa, synthetic construct
  VARIANT 24: note = X is G or C
  VARIANT 2: note = X is A or C
AXAEARRHHQRIEELERRQAEIEXRQAEAEREL

SEQ ID NO: 17 - protein, 33 aa, synthetic construct
AAAEARRHHQRIEELERRQAEIEGRQAEAEREL

SEQ ID NO: 18 - protein, 33 aa, synthetic construct
AAAEARRHHQRIEELERRQAEIECRQAEAEREL

SEQ ID NO: 19 - protein, 33 aa, synthetic construct
ACAEARRHHQRIEELERRQAEIECRQAEAEREL

SEQ ID NO: 20 - protein, 437 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPPGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDAL

SEQ ID NO: 21 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPPGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDAL

SEQ ID NO: 22 - protein, 437 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDAL

SEQ ID NO: 23 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDAL

SEQ ID NO: 24 - protein, 437 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCAL

SEQ ID NO: 25 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCAL

SEQ ID NO: 26 - protein, 437 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCAL

SEQ ID NO: 27 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCAL

SEQ ID NO: 28 - protein, 78 aa, synthetic construct
  VARIANT 1..4: note = optionally absent
LTPLCVTLQCTNVSNNITDDMRGELKNCSFNMTTELRDKKQKVYSLFYRLDVVQINENQGNRTNNTNKEYRLINCNTT

SEQ ID NO: 29 - protein, 24 aa, synthetic construct
  VARIANT 3: note = X is K or C
  VARIANT 6: note = X is P or C
  VARIANT 22: note = X is K or C
SIXQWXPDVSLEPEPLYLCAPXGY

SEQ ID NO: 30 - protein, 11 aa, synthetic construct
NGLYVYLPPVE

SEQ ID NO: 31 - protein, 5 aa, synthetic construct
LGGEG

SEQ ID NO: 32 - protein, 35 aa, synthetic construct
KWEDASVKLYPAAPPTVSPGKTGNWLTGDKVLPTP

SEQ ID NO: 33 - protein, 21 aa, synthetic construct
  VARIANT 14: note = X is D or C
  VARIANT 20: note = X is S or C
MVDQLSEYVQEQFXLALAGXN

SEQ ID NO: 34 - protein, 35 aa, synthetic construct
KWEDASVKLFKACPPTVSPGKTGNYFTGPECPLVP

SEQ ID NO: 35 - protein, 22 aa, synthetic construct
MVDQLSEYVQEQFDLALAGSCN

SEQ ID NO: 36 - protein, 21 aa, synthetic construct
MVDQLSEYVQEQFCLALAGCN

SEQ ID NO: 37 - protein, 37 aa, synthetic construct
  VARIANT 2: note = X is T or C
  VARIANT 4: note = X is A or C
  VARIANT 23: note = X is I or C
SXAXLLKALQATKARADANALEXAEIKERIRETEREL

SEQ ID NO: 38 - protein, 37 aa, synthetic construct
STAALLKALQATKARADANALEIAEIKERIRETEREL

SEQ ID NO: 39 - protein, 37 aa, synthetic construct
SCACLLKALQATKARADANALEICEIKERIRETEREL

SEQ ID NO: 40 - protein, 37 aa, synthetic construct
STACLLKALQATKARADANALEIAEIKERIRETEREL

SEQ ID NO: 41 - protein, 437 aa, synthetic construct
LTPLCVTLQCTNVTNNITDDMRGELKNCSFNMTTELRDKRQKVHALFYKLDIVPINENQNTTYRLINCNTASIKQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSN

SEQ ID NO: 42 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTSIKQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSN

SEQ ID NO: 43 - protein, 437 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSN

SEQ ID NO: 44 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTSICQWPPDVSLEPEPLYLCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSN

SEQ ID NO: 45 - protein, 437 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSN

SEQ ID NO: 46 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTSICQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSN

SEQ ID NO: 47 - protein, 437 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGNTGNWLTGDKVLPTPPNPQEIHLENVTEEFNMWKNNMVDQLSEYVQEQFCLALAGSN

SEQ ID NO: 48 - protein, 437 aa, synthetic construct
LTPLCVTLQCTNVTNNITDDMRGELKNCSFNMTTELRDKRQKVHALFYKLDIVPINENQNTTYRLINCNTASIKQWCPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCN

SEQ ID NO: 49 - protein, 444 aa, synthetic construct
LTPLCVTLQCTNVSNNITDDMRGELKNCSFNMTTELRDKKQKVYSLFYRLDVVQINENQGNRTNNTNKEYRLINCNTTSIKQWCPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCN

SEQ ID NO: 50 - protein, 20 aa, synthetic construct
MDRAKLLLLLLLLLLPQAQA

SEQ ID NO: 51 - protein, 117 aa, synthetic construct
AELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 52 - protein, 612 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTAQIVQWPPDVSLEPEPIYYCAPPGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIEGRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 53 - protein, 619 aa, synthetic construct
MDRAKLLLLLLLLLLPQAQALTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPPGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIEGRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 54 - protein, 612 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTAQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 55 - protein, 619 aa, synthetic construct
MDRAKLLLLLLLLLLPQAQALTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 56 - protein, 612 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTAQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 57 - protein, 619 aa, synthetic construct
MDRAKLLLLLLLLLLPQAQALTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 58 - protein, 612 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 59 - protein, 619 aa, synthetic construct
MDRAKLLLLLLLLLLPQAQALTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 60 - protein, 595 aa, synthetic construct
LTPLCVTLQCTNVTNNITDDMRGELKNCSFNMTTELRDKRQKVHALFYKLDIVPINENQNTTYRLINCNTASIKQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSNGGSGSTAALLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 61 - protein, 602 aa, synthetic construct
LTPLCVTLQCTNVSNNITDDMRGELKNCSFNMTTELRDKKQKVYSLFYRLDVVQINENQGNRTNNTNKEYRLINCNTTSIKQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSNGGSGSTAALLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 62 - protein, 595 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSNCGCGSCACLLKALQATKARADANALEICEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 63 - protein, 602 aa, synthetic construct
LTPLCVTLQCTNVSNNITDDMRGELKNCSFNMTTELRDKKQKVYSLFYRLDVVQINENQGNRTNNTNKEYRLINCNTTSICQWPPDVSLEPEPLYLCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSNCGCGSCACLLKALQATKARADANALEICEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 64 - protein, 595 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSNCGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 65 - protein, 602 aa, synthetic construct
LTPLCVTLQCTNVSNNITDDMRGELKNCSFNMTTELRDKKQKVYSLFYRLDVVQINENQGNRTNNTNKEYRLINCNTTSICQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSNCGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 66 - protein, 595 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGNTGNWLTGDKVLPTPPNPQEIHLENVTEEFNMWKNNMVDQLSEYVQEQFCLALAGSNCGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 67 - protein, 595 aa, synthetic construct
LTPLCVTLQCTNVTNNITDDMRGELKNCSFNMTTELRDKRQKVHALFYKLDIVPINENQNTTYRLINCNTASIKQWCPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCNGGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 68 - protein, 602 aa, synthetic construct
LTPLCVTLQCTNVSNNITDDMRGELKNCSFNMTTELRDKKQKVYSLFYRLDVVQINENQGNRTNNTNKEYRLINCNTTSIKQWCPDVSLEPEPLYLCAPKGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCNGGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 69 - protein, 155 aa, synthetic construct
  VARIANT 1..2: note = optionally absent
MGKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 70 - protein, 155 aa, synthetic construct
  VARIANT 1..2: note = optionally absent
MGKYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 71 - protein, 152 aa, synthetic construct
  VARIANT 1..2: note = optionally absent
MGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 72 - protein, 166 aa, synthetic construct
AILKCKDKKFNGTGPCTNVTTVQCTHGIKPVVSTQLLLNGTLAEEEVMIRSKDIRNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLF

SEQ ID NO: 73 - protein, 450 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDAL

SEQ ID NO: 74 - protein, 443 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCAL

SEQ ID NO: 75 - protein, 443 aa, synthetic construct
LTPLCVTLQCTNVTNNITDDMRGELKNCSFNMTTELRDKRQKVHALFYKLDIVPINENQNTTYRLINCNTASIKQWPPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSN

SEQ ID NO: 76 - protein, 443 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSN

SEQ ID NO: 77 - protein, 443 aa, synthetic construct
LTPLCVTLQCTNVTNNITDDMRGELKNCSFNMTTELRDKRQKVHALFYKLDIVPINENQNTTYRLINCNTASIKQWCPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCN

SEQ ID NO: 78 - protein, 592 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPPGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIEGRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 79 - protein, 598 aa, synthetic construct
TPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPPGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIEGRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 80 - protein, 592 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 81 - protein, 599 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 82 - protein, 592 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 83 - protein, 599 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 84 - protein, 592 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 85 - protein, 599 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 86 - protein, 605 aa, synthetic construct
LTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 87 - protein, 625 aa, synthetic construct
MDRAKLLLLLLLLLLPQAQALTPLCVTLQCTNVSNNITDDMRGELKNCSFNMTTELRDKKQKVYSLFYRLDVVQINENQGNRTNNTNKEYRLINCNTTQIVQWPPDVSLEPEPIYYCAPCGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGDALTGRAPAAAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 88 - protein, 598 aa, synthetic construct
LTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 89 - protein, 618 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 90 - protein, 601 aa, synthetic construct
LTPLCVTLQCTNVTNNITDDMRGELKNCSFNMTTELRDKRQKVHALFYKLDIVPINENQNTTYRLINCNTASIKQWPPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSNGGSGSTAALLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 91 - protein, 621 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTASIKQWPPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFDLALAGSNGGSGSTAALLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 92 - protein, 601 aa, synthetic construct
LTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSNCGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 93 - protein, 621 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTASICQWPPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGSNCGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 94 - protein, 601 aa, synthetic construct
LTPLCVTLQCTNVTNNITDDMRGELKNCSFNMTTELRDKRQKVHALFYKLDIVPINENQNTTYRLINCNTASIKQWCPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCNGGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 95 - protein, 621 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTASIKQWCPDVSLEPEPLYLCAPKGYAILKCKDKKFNGTGPCTNVTTVQCTHGIKPVVSTQLLLNGTLAEEEVMIRSKDIRNNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRNGLYVYLPPVEGVIRCVSNITGLILTRDGGSTDSTTETFRPSGGDMRDNWRSELYKYKVVKILGGEGKWEDASVKLYPAAPPTVSPGKTGNWLTGDKVLPTPPNPQEIHLENVTEEFNMWKNNMVDQLSEYVQEQFCLALAGCNGGSGSTACLLKALQATKARADANALEIAEIKERIRETERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREE

SEQ ID NO: 96 - protein, 4 aa, synthetic construct
GGSG

SEQ ID NO: 97 - protein, 4 aa, synthetic construct
CGCG

SEQ ID NO: 98 - protein, 4 aa, synthetic construct
CGSG

SEQ ID NO: 99 - protein, 4 aa, synthetic construct
  VARIANT 1: note = Can also be C
  VARIANT 3: note = Can also be C
GGSG

SEQ ID NO: 100 - protein, 624 aa, synthetic construct
MDRAKLLLLLLLLLLPQAQALTPLCVTLQCTNVTNNITDDMRGELKNCSFNMTTELRDKRQKVHALFYKLDIVPINENQNTTYRLINCNTAQIVQWPPDVSLEPEPIYYCAPPCYAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNTTDDSNSTSDDNGTITLPCRIKQIINMWQREGQYVYLPPVEGVIRCVSNITGLILTRDGGSTDSTTETFRPSGGDMRDNWRSELYKYKVVKKVGGEGNWEDASVKLFKACPPTVSPGKTGNYFTGPECPLVPPNPQEIHLENVTEEFNMWKNNMVDQVYEDVEYRFRLALGCALTGRAPACAEARRHHQRIEELERRQAEIECRQAEAERELAELAYLLGELAYKLGEYRIAIRAYRIALSRDPGNAEAWYNLGNAYYKQGRYREAIYYFKKALELDPNNAEAWYNLGNAYYERGEYEEAIEYYRKALRLDPNNADAMQNLLNAKMREEGGWELQHHHHHH

SEQ ID NO: 101 - protein, 438 aa, synthetic construct
LTPLCVTLQCTNVTNNITDDMRGELKNCSFNMTTELRDKRQKVHALFYKLDIVPINENQNTTYRLINCNTAAITQACPKVSFEPIPIHYCAPAGFAILKCKDKKFNGTGPCTNVTTVQCTHGIKPVVSTQLLLNGTLAEEEVMIRSEDIRDNAKNILVQFNTTVQINCTRPNNNTRKSIRIGPGQWFYATGDIIGDIRQAHCNVTKATWNETLGKVVKQLRKHFGNNTIIRFANSTGGDLEVTTHSFNCGGEFFYCDTSGLFNSTWISNTTVQGSNSTGSNDTITLPCRIKQIINMWQRIGQAMYAPPIQGVIRCVSNITGLILTRDGGSYDSTTETFRPSGGDMRDNWRSELYKYKVVKIVWKDAETTLFCASDAKAYETKKHNVWATHCCVPTDPNPQEIHLENVTEEFNMWKNNMVEQMHTDIISLWDQSLKPCV

SEQ ID NO: 102 - protein, 447 aa, synthetic construct
LTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIVWKEATTTLFCASDAKAYDTEVHNVWATHACVPTDPNPQEVVLVNVTENFNMWKNDMVEQMHEDIISLWDQSLKPCV